(19) United States
(12) Patent Application Publication
(10) Pub. No.: US 2025/0084085 A1
(43) Pub. Date: Mar. 13, 2025

(54) PROCESS OF MAKING 2-[(3R)-2-[4-AMINO-3-(2-FLUORO-4-PHENOXY-PHENYL)PYRAZOLO[3,4-D]PYRIMIDIN-1-YL]PIPERIDINE-1-CARBONYL]-4-METHYL-4[4-(OXETAN-3-YL)PIPERAZIN-1-YL]-PENT-2-ENENITRILE AND SOLVATE FORMS THEREOF

(71) Applicant: Principia Biopharma Inc., Bridgewater, NJ (US)

(72) Inventors: Myriam Comte, Saint Victor La Coste (FR); Paul Cruciani, Pujaut (FR); Laurent Larquetoux, Nimes (FR); Olivier Latremoliere, Pujaut (FR); Clement Lemaitre, Les Angles (FR); Philippe Ochsenbein, Montpellier (FR); Pierre Versproumy, Les Angles (FR)

(73) Assignee: Principia Biopharma Inc., Bridgewater, NJ (US)

(21) Appl. No.: 18/789,827

(22) Filed: Jul. 31, 2024

Related U.S. Application Data
(60) Provisional application No. 63/530,142, filed on Aug. 1, 2023.

(30) Foreign Application Priority Data
Sep. 8, 2023 (EP) 23196351.3

(51) Int. Cl. C07D 487/04; C07D 305/08

(52) U.S. Cl. CPC C07D 487/04; C07D 305/08

(57) ABSTRACT

Disclosed herein are novel methods for preparing 2-[(3R)-2-[4-amino-3-(2-fluoro-4-phenoxy-phenyl)pyrazolo[3,4-d]pyrimidin-1-yl]piperidine-1-carbonyl]-4-methyl-4[4-(oxetan-3-yl)piperazin-1-yl]-pent-2-enenitrile, including various solvate forms and intermediates thereof. Also disclosed herein are novel solvate forms and intermediates of 2-[(3R)-2-[4-amino-3-(2-fluoro-4-phenoxy-phenyl)pyrazolo[3,4-d]pyrimidin-1-yl]piperidine-1-carbonyl]-4-methyl-4[4-(oxetan-3-yl)piperazin-1-yl]-pent-2-enenitrile.

### CROSS-REFERENCE TO RELATED APPLICATIONS

This application claims priority to U.S. Provisional Application No. 63/530,142, filed on Aug. 1, 2023, and European Application No. 23196351.3, filed on Sep. 8, 2023, the disclosures of each of which are incorporated herein by reference in their entireties for any purpose.

### FIELD

Disclosed herein are novel methods for preparing 2-[(3R)-2-[4-amino-3-(2-fluoro-4-phenoxy-phenyl)pyrazolo[3,4-d]pyrimidin-1-yl]piperidine-1-carbonyl]-4-methyl-4[4-(oxetan-3-yl)piperazin-1-yl]-pent-2-enenitrile, including various solvate forms and intermediates thereof. 2-[(3R)-2-[4-amino-3-(2-fluoro-4-phenoxy-phenyl)pyrazolo[3,4-d]pyrimidin-1-yl]piperidine-1-carbonyl]-4-methyl-4[4-(oxetan-3-yl)piperazin-1-yl]-pent-2-enenitrile, known as rilzabrutinib, and PRN1008, is also referred to herein as the compound of Formula (I), having the structure:

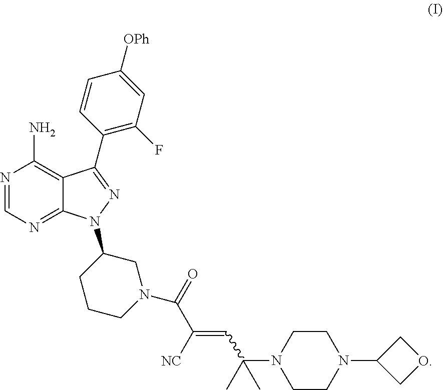 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

Also disclosed herein are various solvate forms and intermediates of 2-[(3R)-2-[4-amino-3-(2-fluoro-4-phenoxy-phenyl)pyrazolo[3,4-d]pyrimidin-1-yl]piperidine-1-carbonyl]-4-methyl-4[4-(oxetan-3-yl)piperazin-1-yl]-pent-2-enenitrile.

### BACKGROUND

The compound described by Formula (I) is an inhibitor of Bruton's tyrosine kinase (BTK) and is useful in the treatment of disorders and conditions mediated by BTK activity. BTK is expressed in most hematopoietic cells, including B cells, mast cells, and macrophages. BTK plays a role in the development and activation of B cells and has been implicated in multiple signaling pathways across a wide range of immune-mediated diseases. BTK activity has been implicated in the pathogenesis of several disorders and conditions, such as B cell-related hematological cancers (e.g., non Hodgkin lymphomia and B cell chronic lymphocytic leukemia) and autoimmune diseases (e.g., rheumatoid arthritis, Sjogren's syndrome, pemphigus, inflammatory bowel disease, lupus, and asthma).

The compound of Formula (I) is disclosed in Example 31 of WO2014/039899. The synthesis of the compound of Formula (I) that is disclosed in WO2014/039899 requires purification by column chromatography and affords a foam upon removal of solvent, which can be crushed to a powder. Crystalline solid forms characterized as Form A, Form B, and an acetonitrile solvate (referred to also as crystalline Form C) of the compound of Formula (I), as well as processes of preparation thereof, are disclosed in WO2021/150723.

For a compound to be suitable for use as a therapeutic agent, its synthesis should be amenable to large scale manufacturing and isolation, and the physical properties of the compound should be such that they do not negatively impact the effectiveness or cost of a formulated active ingredient. The present disclosure addresses such needs.

### SUMMARY

Disclosed herein are novel solvate forms of the compound of Formula (I) and methods of making the same.

It has been surprisingly found that an intermediate solvate form, preferably an ester solvate or a carbonate solvate, of the compound of Formula (I) can be used to efficiently form a suitable crystalline final form of the compound of Formula (I). Useful methods for producing an intermediate solvate form of the compound of Formula (I) that can provide suitable crystalline forms of the compounds of Formula (I), e.g. as crystalline Form A (for example, characterized by an X-ray powder diffractogram having a signal at at least three two-theta values chosen from 5.6±0.2, 12.7±0.2, 16.5±0.2, 17.0±0.2, 17.7±0.2, 18.7±0.2, 19.2±0.2, 20.7±0.2, 22.2±0.2, and 24.4±0.2) or Form B (for example, characterized by an X-ray powder diffractogram having a signal at at least three two-theta values chosen from 10.8±0.2, 15.3±0.2, 16.3±0.2, 17.9±0.2, 18.4±0.2, 18.7±0.2, 22.0±0.2, and 22.9±0.2), have also been found. In at least one embodiment the crystalline form is Form B. The intermediate solvate forms can exhibit high crystallinity and impart a purification effect to the final non-solvated crystalline solid forms of the compounds of Formula (I).

Intermediate solvate forms of these two solvents have the remarkable property to be efflorescent (a chemical that has a solvent associated with its molecules, which when exposed to air, loses the solvent through evaporation) and can be desolvated at ambient temperature and ambient pressure to lead to non-solvated crystalline solid forms, including Form A, Form B, and a mixture of Form A and Form B of the compound of Formula (I). This efflorescence property linked to the volatility of the solvents allows the final crystalline form to have a low residual content of solvent post-desolvation. A polishing step using a class III solvent can be applied to obtain Form B of the compound of Formula (I). This polishing step selectively dissolves the crystalline Form A over Form B and improves the crystallinity, the crystalline purity, and the stability of the obtained crystalline Form B of the compound of Formula (I).

In another aspect, disclosed herein are a methylacetate solvate form of the compound of Formula (I) and a dimethylcarbonate solvate form of the compound of Formula (I).

In a related aspect, provided herein are methods for preparing a compound of Formula (I) and intermediates in the synthesis thereof.

In a further aspect, provided herein are methods for preparing a compound of Formula (I) in one or more crystalline forms.

The present disclosure relates to a method of preparing a compound of Formula (I):

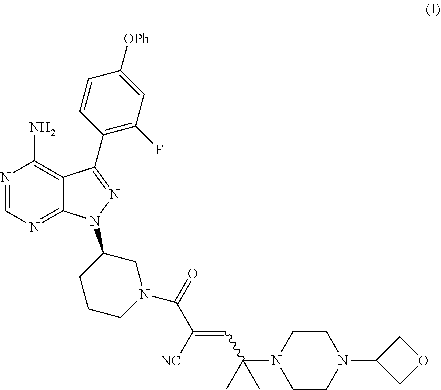 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

or a salt thereof, comprising:
reacting a compound of Formula (I-g):

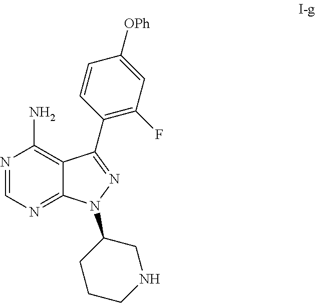 [SMILES: Nc1ncnc2c1c(-c1ccc(Oc3ccccc3)cc1F)nn2[C@@H]1CCCNC1]

or a salt thereof, with a compound of Formula (I-n):

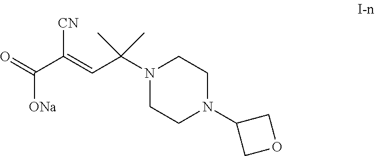

or a salt thereof, with amide-coupling reagent, organic base, and acid in organic solvent to form the compound of Formula (I).

The present disclosure also relates to a method of preparing a compound of Formula (I-n):

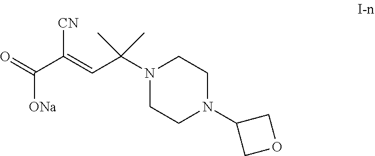

or a salt thereof, by reacting a compound of Formula (I-l):

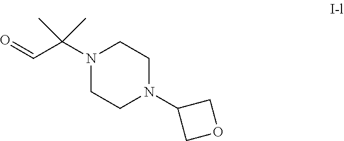

or a salt thereof, with a compound of Formula (I-m):

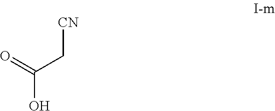

or a salt thereof, with inorganic or organic base in organic solvent to form the compound of Formula (I-n).

The present disclosure further relates to a method of preparing a compound of Formula (I-g):

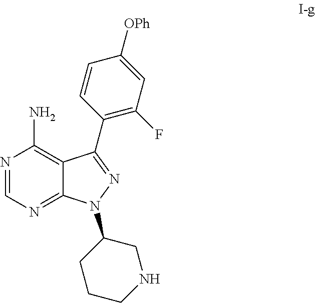 [SMILES: Nc1ncnc2c1c(-c1ccc(Oc3ccccc3)cc1F)nn2[C@@H]1CCCNC1]

or a salt thereof, by reacting a compound of Formula (I-f):

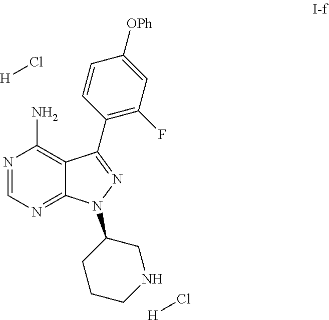

or a salt thereof, with base in organic solvent and water to form the compound of Formula (I-g).

The present disclosure relates to a compound of Formula (I-n):

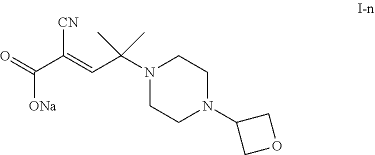

or a salt thereof.

The present disclosure also relates to a compound of Formula (I-g):

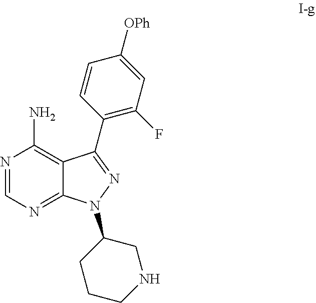 [SMILES: Nc1ncnc2c1c(-c1ccc(Oc3ccccc3)cc1F)nn2[C@@H]1CCCNC1]

or a salt thereof, for example, a compound of Formula (I-f):

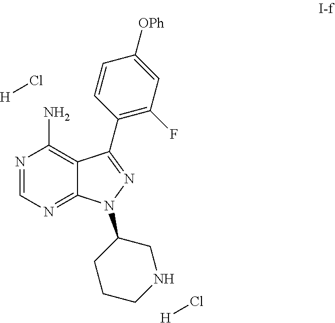

or a salt thereof.

The present disclosure relates to an ester solvate of a compound of Formula (I-E):

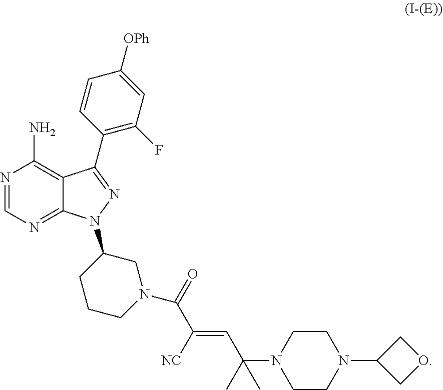 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

The present disclosure relates to a carbonate solvate of a compound of Formula (I-E):

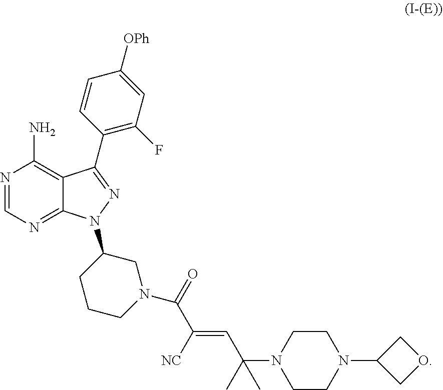 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

### BRIEF DESCRIPTION OF THE DRAWINGS

FIG. 1 shows an X-ray powder diffractogram (XRPD) of the methylacetate (MAC) solvate of the compound of Formula (I-(E)) and a mixture of crystalline Form B/Form A 90/10 of the compound of Formula (I-(E)) after drying the MAC solvate of the compound of Formula (I-(E)).

FIG. 2 shows XRPD patterns of a mixture of crystalline Form B/Form A 90/10 of the compound of Formula (I-(E)) after drying and crystalline Form B of the compound of Formula (I-(E)) after the polishing step with ethanol.

FIG. 3 shows an XRPD pattern of a mixture of crystalline Form A and crystalline Form B of the compound of Formula (I-(E)) obtained after 2 h of filtration and drying a dimethylcarbonate (DMC) solvate of the compound of Formula (I-(E)) and an XRPD pattern after the polishing step with IPAC that yields crystalline Form B.

FIG. 4 shows XRPD pattern of crystalline Form B of the compound of Formula (I-(E)) obtained directly without any polishing step by applying 50 bar CO₂ on the suspension of the methylacetate solvate for one night. which is compared to the XRPD pattern of reference crystalline Form B of the compound of Formula (I-(E)).

FIG. 5 shows an Oak Ridge Thermal Ellipsoid Plot (ORTEP) representation of the crystalline structure of the compound of Formula (I-(E)) crystallized in methylacetate.

FIG. 6 shows an XRPD pattern of the MAC solvate of the compound of Formula (I-(E)) compared to the XRPD pattern of reference crystalline Form B of the compound of Formula (I-(E)).

FIG. 7 shows the XRPD pattern of the AcOMe solvate of the compound of Formula (I-(E)) after Pawley refinement.

FIG. 8 shows the unit cell parameters for the AcOMe solvate of the compound of Formula (I-(E)) at room temperature (298 K).

FIG. 9 shows crystals of MAC solvate observed by polarized optical microscopy.

FIG. 10 shows micro cracks on the surface of crystals of MAC solvate observed by polarized optical microscopy.

FIG. 11 shows an XRPD pattern of the DMC solvate of the compound of Formula (I-(E)).

FIG. 12 shows the unit cell parameters for the DMC solvate of the compound of Formula (I-(E)) at room temperature (298 K).

FIG. 13 shows a TG/DSC data of DMC solvate of the compound of Formula (I-(E)).

FIG. 14 shows XRPD patterns of the DMC solvate of the compound of Formula (I-(E)) after desolvation and after desolvation and polishing to form a mixture of crystalline Form A and Form B of the compound of Formula (I-(E)) and crystalline Form B of the compound of Formula (I-(E)), respectively.

FIG. 15 shows crystals of the DMC solvate of the compound of Formula (I-(E)) observed by polarized optical microscopy.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying figures, which are incorporated in and constitute a part of this specification, illustrate several embodiments and together with the description, serve to explain the principles described herein.

### DETAILED DESCRIPTION

### Definitions

Unless otherwise stated, the following terms used in the specification and claims are defined for the purposes of this disclosure and have the following meaning.

As used herein, “a” or “an” entity refers to one or more of that entity, e.g., “a compound” refers to one or more compounds or at least one compound unless stated otherwise. As such, the terms “a” (or “an”), “one or more” and “at least one” are used interchangeably herein.

As used herein, the term “about” means approximately, in the region of, roughly or around. When the term “about” is used in conjunction with a numerical range it modifies that range by extending the boundaries above and below the numerical values set forth. In general, the term “about” is used herein to modify a numerical value above and below the stated value by a variance of 5%.

As used herein, “2-[(3R)-2-[4-amino-3-(2-fluoro-4-phenoxy-phenyl) pyrazolo[3,4-d]pyrimidin-1-yl]piperidine-1-carbonyl]-4-methyl-4[4-(oxetan-3-yl) piperazin-1-yl]-pent-2-enenitrile”, “PRN1008”, “rilzabrutinib” and “the compound of Formula (I),” are used interchangeably to refer to a compound having the following structure:

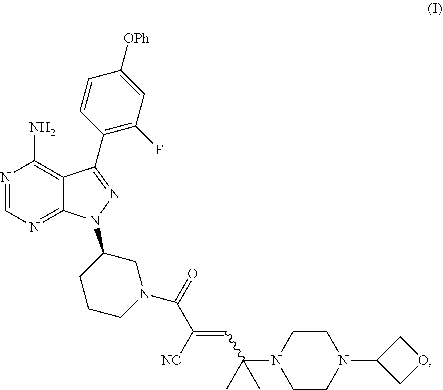 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also known as 2-[(3R)-2-[4-amino-3-(2-fluoro-4-phenoxy-phenyl) pyrazolo[3,4-d]pyrimidin-1-yl]piperidine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, 1-piperidinepropanenitrile, 3-[4-amino-3-(2-fluoro-4-phenoxyphenyl)-1H-pyrazolo[3,4-d]pyrimidin-1-yl]-α-[2-methyl-2-[4-(3-oxetanyl)-1-piperazinyl]propylidene]-β-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile, and also by the International Nonproprietary Names for Pharmaceutical Substances (INN) as published by the World Health Organization (https://cdn.who.int/media/docs/default-source/international-nonproprietary-names-(inn)/pl121.pdf?sfvrsn=69617906_15&download=true) having the following structure:

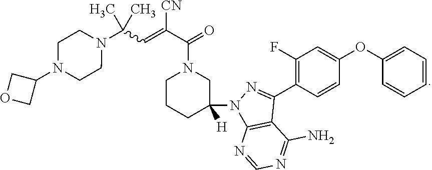 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

The compound of Formula (I) includes E and Z isomers, as indicated by the wavy bond in the structure shown above. The compound of Formula (I) may be present as a salt form.

A dose of the (E) isomer of the compound of Formula (I) may contain the corresponding (Z) isomer as an impurity in less than about 5% by weight, for example less than about 2% by weight, such as less than 1% by weight; a dose of the (Z) isomer of the compound of Formula (I) may contain the corresponding (E) isomer as an impurity in less than about 5% by weight, for example less than about 2% by weight, such as less than 1% by weight. When the compound of Formula (I) is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 2% by weight.

In some embodiments, the molar ratio of the (E) to (Z) isomer of the compound of Formula (I) is 8:2. In some embodiments, the molar ratio of the (E) to (Z) isomer of the compound of Formula (I) is 9:1. In some embodiments, the molar ratio of the (E) to (Z) isomer of the compound of Formula (I) is 98:2. In some embodiments, the molar ratio of the (E) to (Z) isomer of the compound of Formula (I) is 99:1.

In some embodiments, the compound of Formula (I) is provided as the (E) isomer. In some embodiments, the compound of Formula (I) is provided as the (Z) isomer. In some embodiments, the compound of Formula (I) is provided as a mixture of (E) and (Z) isomers.

In some embodiments, the compound of Formula (I) is a compound of Formula (I-(E)), the E isomer of the compound of Formula (I). In some embodiments, the compound of Formula (I-(E)) has the following structure:

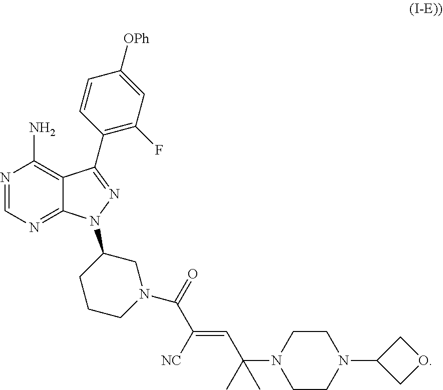 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, the compound of Formula (I-(E)) is 95% pure, such as 96% pure, 97% pure, 98% pure, 99% pure, or 100% pure.

In some embodiments, the compound of Formula (I) is a compound of Formula (I-(Z)), the Z isomer of the compound of Formula (I). In some embodiments, the compound of Formula (I-(Z)) has the following structure:

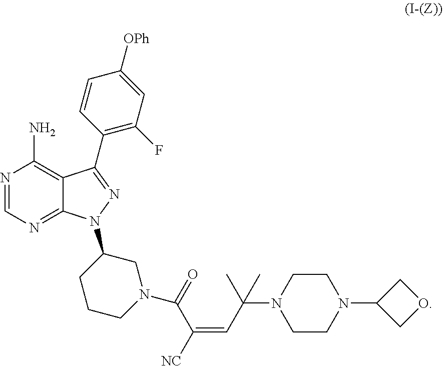 [SMILES: CC(C)(/C=C(/C#N)C(=O)N1CCC[C@@H](n2nc(-c3ccc(Oc4ccccc4)cc3F)c3c(N)ncnc32)C1)N1CCN(C2COC2)CC1]

The compound of Formula (I), or intermediates in the synthesis thereof, may also exist in the form of solvates, in other words in the form of associations or combinations with one or more molecules of solvent. These solvates include more particularly hydrates, where the solvent is water. As used herein, the terms “solvate form” and “solvate” interchangeably refer to a physical association of a compound with one or more solvent molecules. Solvate forms can be identified and distinguished from each other by at least one characterization technique including, e.g., X-ray powder diffraction (XRPD), differential scanning calorimetry (DSC), and thermogravimetric (TG) analysis.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use.

As used herein, the term “treat”, “treating” or “treatment”, when used in connection with a disorder or condition, includes any effect e.g., lessening, reducing, modulating, ameliorating, or eliminating, that results in improvement of the disorder or condition. Improvements in or lessening the severity of any symptom of the disorder or condition can be readily assessed according to standard methods and techniques known in the art.

As used herein, the term “XRPD” refers to the analytical characterization method of X-ray powder diffraction. XRPD patterns can be recorded at ambient conditions in transmission or reflection geometry using a diffractometer.

As used herein, “wet methylacetate solvate” and “wet cake” refers to a solvate that was dried until having between about 10% and about 12%, or about 10%, of its total weight as remaining traces of solvent. In an embodiment, the terms refer to a solvate that was dried until having 11%±1% of its total weight as remaining traces of solvent.

As used herein, Class III solvents refer to solvents with low toxic potential and of relatively low risk to human health, as determined by FDA guidance. Available data indicate that those solvents are less toxic in acute or short-term studies and negative in genototoxicity studies.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the compounds and/or methods disclosed herein.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

Although various features of the compounds and methods disclosed herein may be described in the context of a single embodiment, the features may also be provided separately or in any suitable combination. Conversely, although the compounds and methods disclosed herein may be described in the context of separate embodiments for clarity, the compounds and methods disclosed herein may also be implemented in a single embodiment.

### Methods of Preparation

In accordance with the current disclosure, the compound of Formula (I) can be prepared by one or more of the following processes. The processes described herein can be combined in any suitable order, as would be understood by a person of skill in the relevant art.

### Compound of Formula (I)

Described herein is a method of preparing a compound of Formula (I):

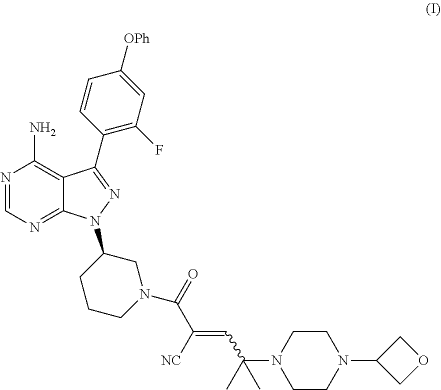 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

or a salt thereof, comprising:
reacting a compound of Formula (I-g):

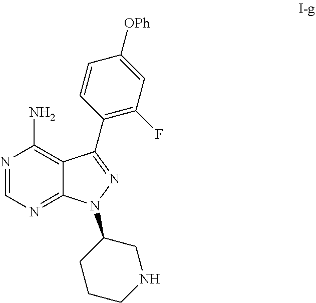 [SMILES: Nc1ncnc2c1c(-c1ccc(Oc3ccccc3)cc1F)nn2[C@@H]1CCCNC1]

or a salt thereof, with a compound of Formula (I-n):

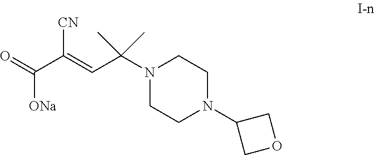

or a salt thereof, with amide-coupling reagent, organic base, and acid in organic solvent to form the compound of Formula (I).

In some embodiments, the amide-coupling reagent is propylphosphonic anhydride.

In some embodiments, the organic base is N-methylmorpholine, triethylamine, or diisopropylamine. In some embodiments, the organic base is N-methylmorpholine. In some embodiments, the organic base is triethylamine. In some embodiments, the organic base is diisopropylamine.

In some embodiments, the acid is hydrochloric acid, methanesulfonic acid, paratoluenesulfonic acid, or chlorotoluenesulfonic acid. In some embodiments, the acid is hydrochloric acid. In some embodiments, the acid is methanesulfonic acid. In some embodiments, the acid is chlorotoluenesulfonic acid.

In some embodiments, the organic solvent comprises dichloromethane, dichloroethane, acetonitrile, chloroform, 2-methyltetrahydrofuran, tetrahydrofuran, methyl tert-butyl ether, toluene, chlorobenzene, anisole (methoxybenzene), methanol, ethanol, or isopropanol. In some embodiments, the organic solvent comprises dichloromethane and isopropanol. In some embodiments, the organic solvent comprises dichloromethane. In some embodiments, the organic solvent comprises isopropanol.

In some embodiments, the reaction is carried out at a temperature ranging from 20 to 50° C. In some embodiments, the reaction is carried out at a temperature ranging from 25 to 40° C. In some embodiments, the reaction is carried out at a temperature of 30° C.

In some embodiments, the reaction is carried out at a pH ranging from 3.5 to 5.5. In some embodiments, the reaction is carried out at a pH ranging from 4 to 5. In some embodiments, the reaction is carried out at a pH of 4.5.

### Compound of Formula (I-n)

Also described herein is a method of preparing a compound of Formula (I-n):

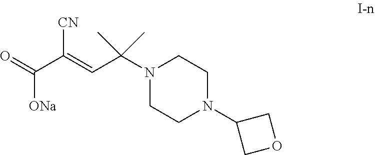

or a salt thereof, by reacting a compound of Formula (I-l):

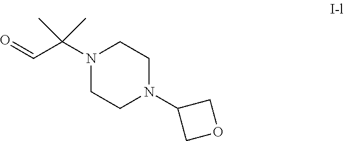

or a salt thereof, with a compound of Formula (I-m):

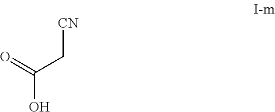

or a salt thereof, with with inorganic or organic base in organic solvent to form the compound of Formula (I-n).

In some embodiments, the inorganic base is sodium hydroxide, lithium hydroxide, or potassium hydroxide. In some embodiments, the inorganic base is sodium hydroxide. In some embodiments, the inorganic base is lithium hydroxide. In some embodiments, the inorganic base is is sodium hydroxide.

In some embodiments, the organic base is sodium methoxide or cesium methoxide. In some embodiments, the organic base is sodium methoxide. In some embodiments, the organic base is cesium methoxide.

In some embodiments, the organic solvent is alcohol. In some embodiments, the alcohol is methanol, ethanol, or propanol. In some embodiments, the alcohol is methanol. In some embodiments, the alcohol is ethanol. In some embodiments, the alcohol is propanol.

In some embodiments, the reaction is carried out at a temperature ranging from 30 to 70° C. In some embodiments, the reaction is carried out at a temperature ranging from 40 to 60° C. In some embodiments, the reaction is carried out at a temperature of 50° C.

### Compound of Formula (I-g)

Further described herein is a method of preparing a compound of Formula (I-g):

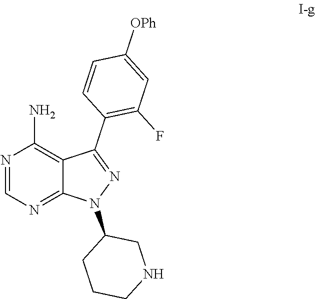 [SMILES: Nc1ncnc2c1c(-c1ccc(Oc3ccccc3)cc1F)nn2[C@@H]1CCCNC1]

or a salt thereof, by reacting a compound of Formula (I-f):

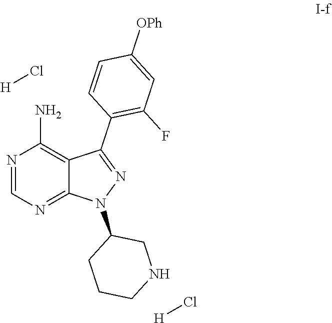

or a salt thereof, with base in organic solvent and water to form the compound of Formula (I-g).

In some embodiments, the base is sodium hydroxide, lithium hydroxide, or potassium hydroxide. In some embodiments, the base is sodium hydroxide. In some embodiments, the base is lithium hydroxide. In some embodiments, the base is potassium hydroxide.

In some embodiments, the organic solvent is alcohol. In some embodiments, the alcohol is methanol, ethanol, or propanol. In some embodiments, the alcohol is methanol. In some embodiments, the alcohol is ethanol. In some embodiments, the alcohol is propanol.

In some embodiments, the reaction is carried out at a temperature ranging from 40 to 70° C. In some embodiments, the reaction is carried out at a temperature ranging from 45 to 60° C. In some embodiments, the reaction is carried out at a temperature of 50° C.

In some embodiments, the reaction is carried out at a pH ranging from 6.0-13.5. In some embodiments, the reaction is carried out at a pH ranging from 7.3-12.5.

In some embodiments, the method of preparing a compound of Formula (I-g) further comprising preparing a compound of Formula (I-f):

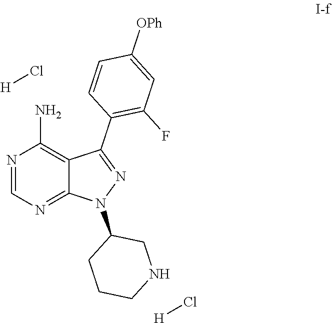

or a salt thereof, by reacting a compound of Formula (I-e):

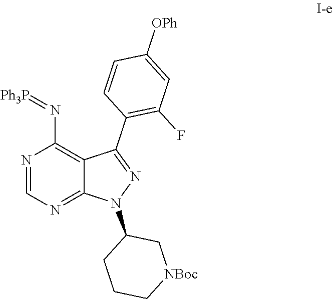

or a salt thereof, with trimethylsilyl chloride (TMSCl).

In some embodiments, the method further comprising preparing a compound of Formula (I-e) or a salt thereof, by reacting a compound of Formula (I-c):

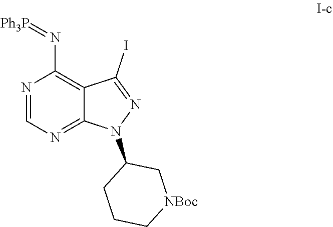

or a salt thereof, with a compound of Formula (I-d):

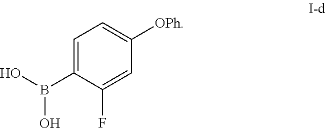 [SMILES: OB(O)c1ccc(Oc2ccccc2)cc1F]

In some embodiments, the reaction of the compound of Formula (I-c) and the compound of Formula (I-d) is a Suzuki reaction. In some embodiments, the reaction of the compound of Formula (I-c) and the compound of Formula (I-d) occurs in the presence of a catalyst. In some embodiments, the catalyst is 1,1′-bis(diphenylphosphino)ferrocene]dichloropalladium(II) (Pd(dppf)Cl₂).

In some embodiments, the compound of Formula (I-g) is synthesized from the compound of Formula (I-c) without isolating the compounds of Formula (I-e) and (I-f).

### Compound of Formula (I-(E))

Also described herein is a method of preparing a compound of Formula (I), or a salt thereof, wherein the compound of Formula (I), or a salt thereof, is at least 95% by weight a compound of Formula (I-(E)):

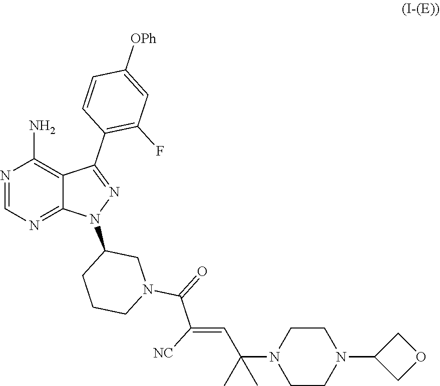 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

or a salt thereof.

### Solvate of a Compound of Formula (I)

In another accordance with the current disclosure, a solvate a compound of Formula (I) can be prepared by one or more of the following processes. The processes described herein can be combined in any suitable order, as would be understood by a person of skill in the relevant art.

### Methylacetate Solvate of a Compound of Formula (I)

Described herein is a method of preparing a methylacetate solvate of a compound of Formula (I-E):

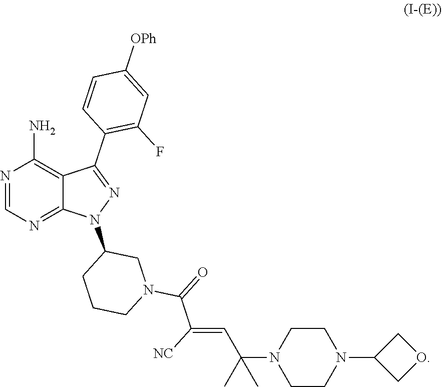 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, crystalline Form B of the compound of Formula (I-(E)) is dissolved in methylacetate to form a solution. In some embodiments, the solution is heated at a temperature ranging from 30 to 50° C. followed by cooling to a temperature ranging from 0 to 15° C. In some embodiments, the solution is heated at a temperature of 40° C. followed by cooling to a temperature ranging from 0 to 10° C.

In some embodiments, the amorphous compound of Formula (I-(E)) is dissolved in methylacetate to form a solution. In some embodiments, the solution is seeded with crystalline Form B of the compound of Formula (I-(E)) to form a suspension followed by stirring at a temperature ranging from 20 to 30° C.

Also described herein is a method of preparing a dimethylcarbonate solvate of a compound of Formula (I-E):

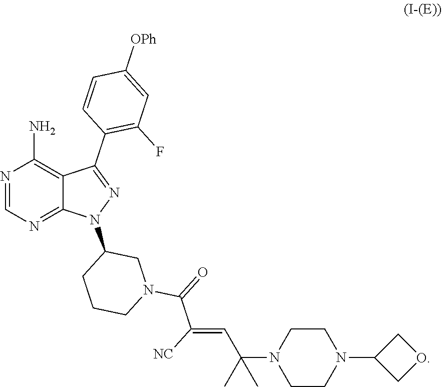 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, crystalline Form C of the compound of Formula (I-(E)) is dissolved in dimethylcarbonate to form a solution. In some embodiments, the solution is frozen, thawed, and stirred at a temperature ranging from 20 to 30° C. to induce particles that stay suspended in solution and do not sediment.

In some embodiments, the amorphous compound of Formula (I-(E)) is dissolved in dimethylcarbonate to form a solution. In some embodiments, the solution is seeded with crystalline Form B of the compound of Formula (I-(E)) to form a suspension. In some embodiments, the suspension is frozen at a temperature ranging from −10 to −30° C. In some embodiments, the suspension is frozen at a temperature of −18° C. In some embodiments, the frozen solution is thawed and stirred at a temperature ranging from 20 to 30° C.

### Crystalline Form of a Compound of Formula (I)

In another accordance with the current disclosure, a crystalline Form of a compound of Formula (I) can be prepared by one or more of the following processes. The processes described herein can be combined in any suitable order, as would be understood by a person of skill in the relevant art.

Described herein is a method of preparing a crystalline form of a compound of Formula (I-E):

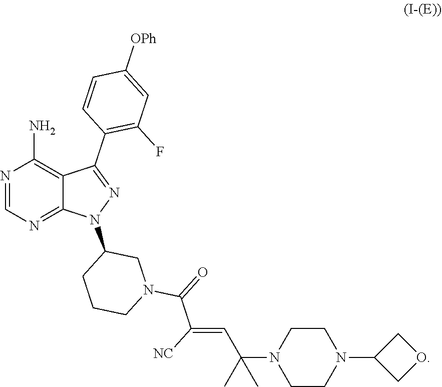 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, a mixture of crystalline Form A and crystalline Form B of a compound of Formula (I-(E)) is prepared by crystallizing the methylacetate solvate of the compound of Formula (I-(E)) in 2-10% dichloromethane in methylacetate with seeds of crystalline Form B, a mixture of crystalline Form A and crystalline Form B, or wet methylacetate solvate of the compound of Formula (I-(E)) to form a suspension. In some embodiments, a mixture of crystalline Form A and crystalline Form B of a compound of Formula (I-(E)) is prepared by crystallizing the methylacetate solvate of the compound of Formula (I-(E)) in 5-8% dichloromethane in methylacetate with seeds of crystalline Form B, a mixture of crystalline Form A and crystalline Form B, or wet methylacetate solvate of the compound of Formula (I-(E)) to form a suspension. In some embodiments, a mixture of crystalline Form A and crystalline Form B of a compound of Formula (I-(E)) is prepared by crystallizing the methylacetate solvate of the compound of Formula (I-(E)) in 4% dichloromethane in methylacetate with seeds of crystalline Form B, a mixture of crystalline Form A and crystalline Form B, or wet methylacetate solvate of the compound of Formula (I-(E)) to form a suspension. In some embodiments, the suspension is cooled, filtered, and dried to yield a mixture of crystalline Form A and crystalline Form B of the compound of Formula (I-(E)). In some embodiments, the drying process is carried out at a pressure ranging from 0 to 800 mbars and a temperature ranging from 0 to 70° C. In some embodiments, the drying process is carried out at a pressure ranging from 50 to 600 mbars and a temperature ranging from 0 to 50° C.

In some embodiments, crystalline Form B of the compound of Formula (I-(E)) is prepared by partically dissolving the suspension comprising a mixture of crystalline Form A and crystalline Form B of the compound of Formula (I-(E)) in ethanol and subjecting it to heating and cooling cycles at a temperature ranging from 0 to 50° C. In some embodiments, crystalline Form B of the compound of Formula (I-(E)) is prepared by partically dissolving the suspension comprising a mixture of crystalline Form A and crystalline Form B of the compound of Formula (I-(E)) in ethanol and subjecting it to heating and cooling cycles at a temperature ranging from 20 to 43° C. In some embodiments, the mixture of crystalline Form A and crystalline Form B of the compound of Formula (I-(E)) is further cooled to a temperature of 0° C.

In some embodiments, crystalline Form B of the compound of Formula (I-(E)) is prepared by partically dissolving the suspension comprising a mixture of crystalline Form A and crystalline Form B of the compound of Formula (I-(E)) in isopropylacetate and subjecting it to to heating, cooling, and stirring. In some embodiments, the heating is carried out in 3-5 steps at a temperature ranging from 20 to 70° C. for 1 to 3 hours. In some embodiments, the heating is carried out in 4 steps at a temperature ranging from 30 to 60° C. for 2 hours. In some embodiments, the cooling is carried out at a temperature ranging from 15 to 40° C. within 1 to 3 hours followed by stirring for 8-16 hours. In some embodiments, the cooling is carried out at a temperature ranging from 20 to 30° C. within 2 hours followed by stirring for 12 hours.

In some embodiments, crystalline Form B of the compound of Formula (I-(E)) is prepared by subjecting the suspension comprising a mixture of crystalline Form A and crystalline Form B of the compound of Formula (I-(E)) to pressure. In some embodiments, the pressure ranges from 30 to 70 bars. In some embodiments, the pressure is 50 bars. In some embodiments, the pressure is applied with inert gas. In some embodiments, the pressure is applied with carbon dioxide, N₂, or Ar.

### Compounds

In some embodiments, provided herein is a compound selected from the compounds in Table 1, or a salt thereof. In some embodiments, provided is a solvate of a compound selected from the compounds in Table 1, or a salt thereof. In some embodiments, provided is a crystalline form of a compound selected from the compounds in Table 1, or a salt thereof.

Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound of | |
|---|---|
| Formula No. | Structure |
| I | 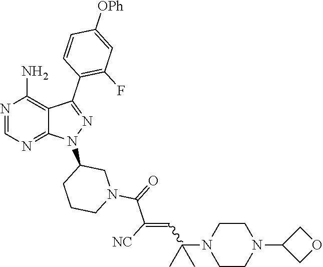 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1] |
| I-(E) | 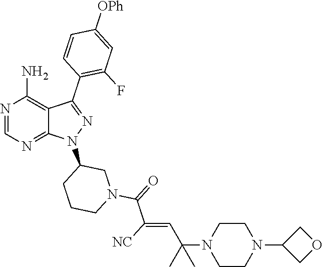 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1] |
| I-(Z) | 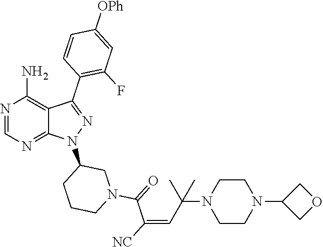 [SMILES: CC(C)(/C=C(/C#N)C(=O)N1CCC[C@@H](n2nc(-c3ccc(Oc4ccccc4)cc3F)c3c(N)ncnc32)C1)N1CCN(C2COC2)CC1] |
| I-a | 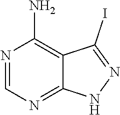 [SMILES: Nc1ncnc2[nH]nc(I)c12] |
| I-b | 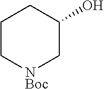 [SMILES: CC(C)(C)OC(=O)N1CCC[C@H](O)C1] |
| I-c | 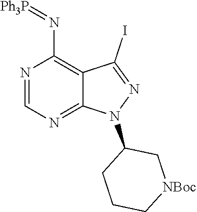 |
| I-d | 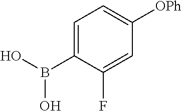 [SMILES: OB(O)c1ccc(Oc2ccccc2)cc1F] |
| I-e | 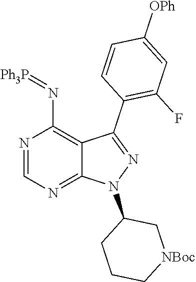 |
| I-f | 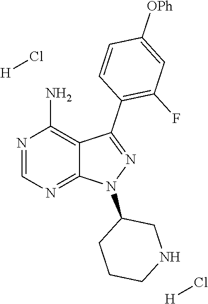 |
| I-g | 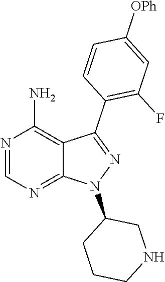 [SMILES: Nc1ncnc2c1c(-c1ccc(Oc3ccccc3)cc1F)nn2[C@@H]1CCCNC1] |
| I-h | 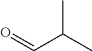 |
| I-i | 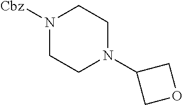 [SMILES: O=C(OCc1ccccc1)N1CCN(C2COC2)CC1] |
| I-j | 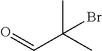 |
| I-k | 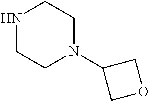 |
| I-l | 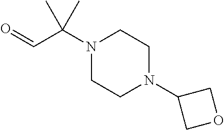 |
| I-m | 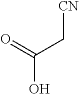 |
| I-n | 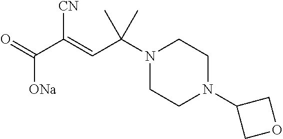 |

or a salt thereof.

In some embodiments, provided herein is a compound of Formula (I-n):

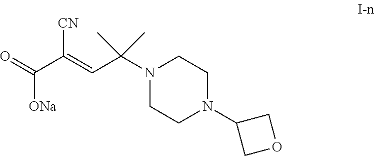

or a salt thereof.

In some embodiments, provided herein is a compound of Formula (I-g):

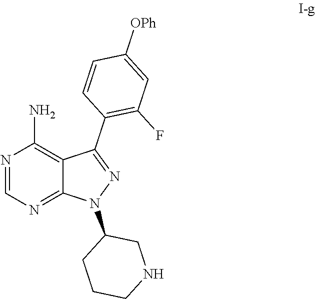 [SMILES: Nc1ncnc2c1c(-c1ccc(Oc3ccccc3)cc1F)nn2[C@@H]1CCCNC1]

or a salt thereof, for example, a compound of Formula (I-f):

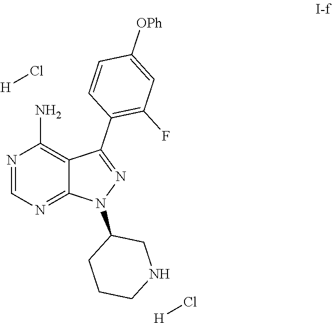

or a salt thereof.

### Solvates

In some embodiments, provided herein is an ester solvate of the compound of Formula (I).

In some embodiments, provided herein is an ester solvate of the compound of Formula (I-(E)):

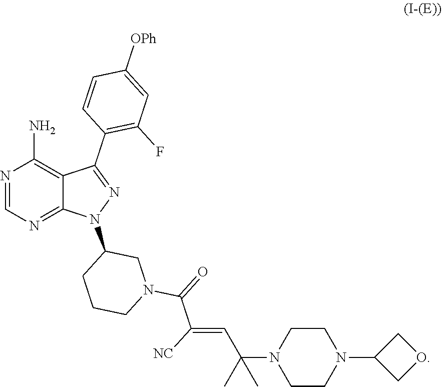 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, the ester solvate is a methylacetate solvate. In some embodiments, the methylacetate solvate has an XRPD pattern comprising one or more peaks chosen from about 4.676° 2θ, 10.798° 2θ, 14.027° 2θ, 14.280° 2θ, 16.198° 2θ, and 16.704° 2θ.

In some embodiments, provided herein is a carbonate solvate of the compound of Formula (I).

In some embodiments, provided herein is a carbonate solvate of the compound of Formula (I-(E)):

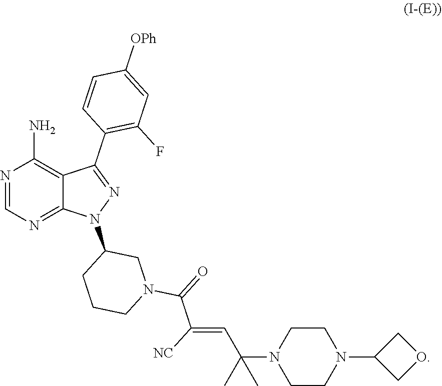 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

In some embodiments, the carbonate solvate is a dimethylcarbonate solvate. In some embodiments, the dimethylcarbonate solvate has an XRPD pattern comprising one or more peaks chosen from about 4.765° 2θ, 9.519° 2θ, 10.668° 2θ, 10.829° 2θ, 14.264° 2θ, and 16.976° 2θ.

### EXAMPLES

The following Examples are presented by way of illustration, not limitation. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatographic purification, with a solution of an acid (for example, with aqueous HCl).

The following abbreviations are used herein.

### Abbreviations

2-MeTHF: 2-methyltetrahydrofuran
AcOMe or MAC: methyl acetate
DCM: dichloromethane
DIAD: diisopropyl azodicarboxylate
DIPEA: N,N-diisopropylethylamine
DMC: dimethylcarbonate
eq: equivalents
EtOH: ethanol
IPAC: isopropylacetate
h or hr: hour(s)
K: kelvin
LCMS: liquid chromatography mass spectrometry
MeOH: methanol
MeSO₃H: methanesulfonic acid
MTBE: methyl tert-butyl ether
NMM: N-methyl-morpholine
NMR: nuclear magnetic resonance
Pd(dppf)Cl₂: [1,1′-bis(diphenylphosphino)ferrocene]dichloropalladium(II)
Ph: phenyl
Ph₃P: triphenylphosphine
RT or rt: room temperature
T₃P: propylphosphonic anhydride
UO: mass or volume equivalent to the reference material
THF: tetrahydrofuran
V: volume per starting material (e.g., 10V solvent for 1 g of starting material=10 mL)
XRPD: X-ray (powder) diffraction

In accordance with the current disclosure, the compound of Formula (I-(E)) can be prepared by the following process.

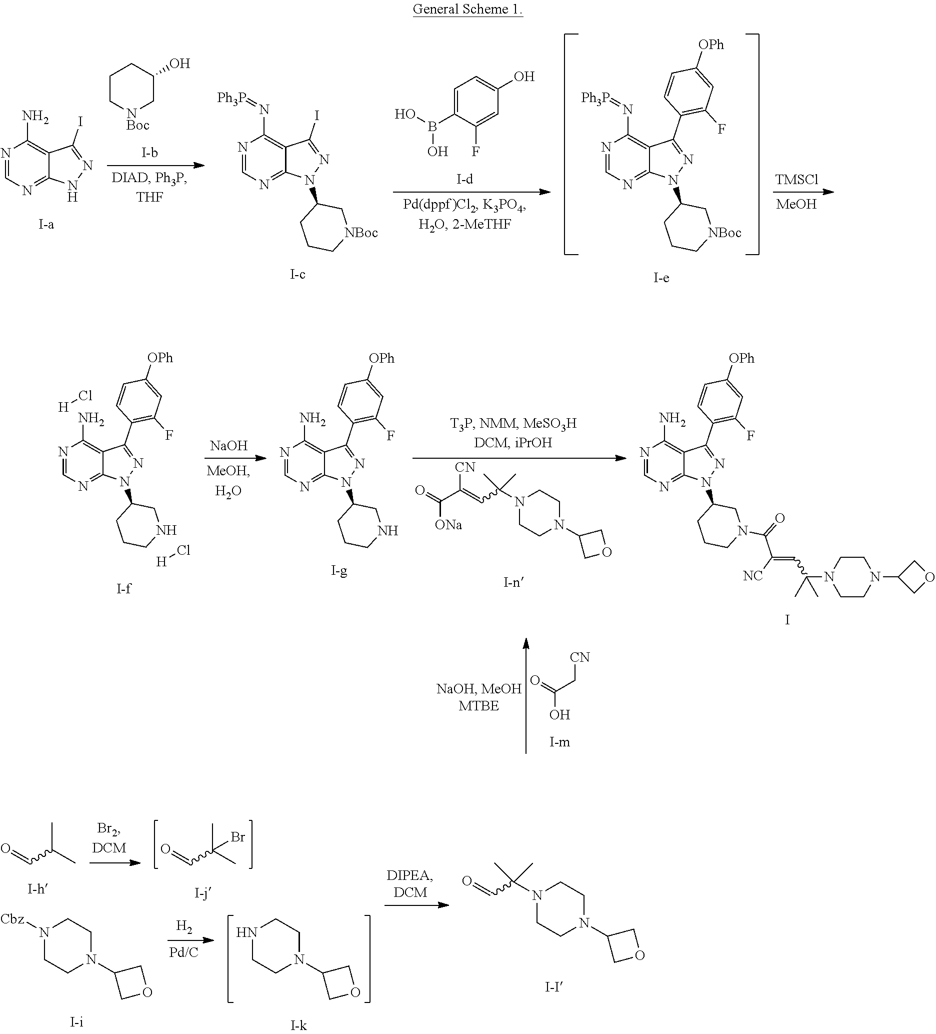

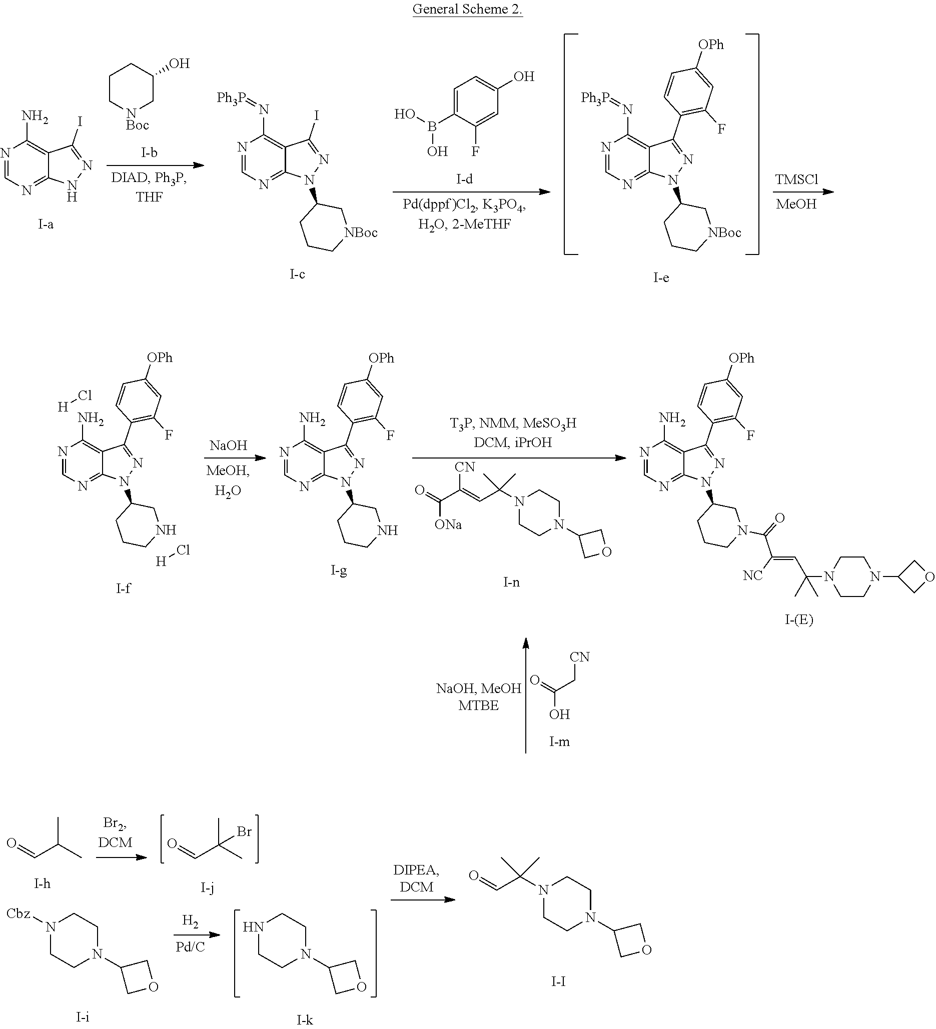

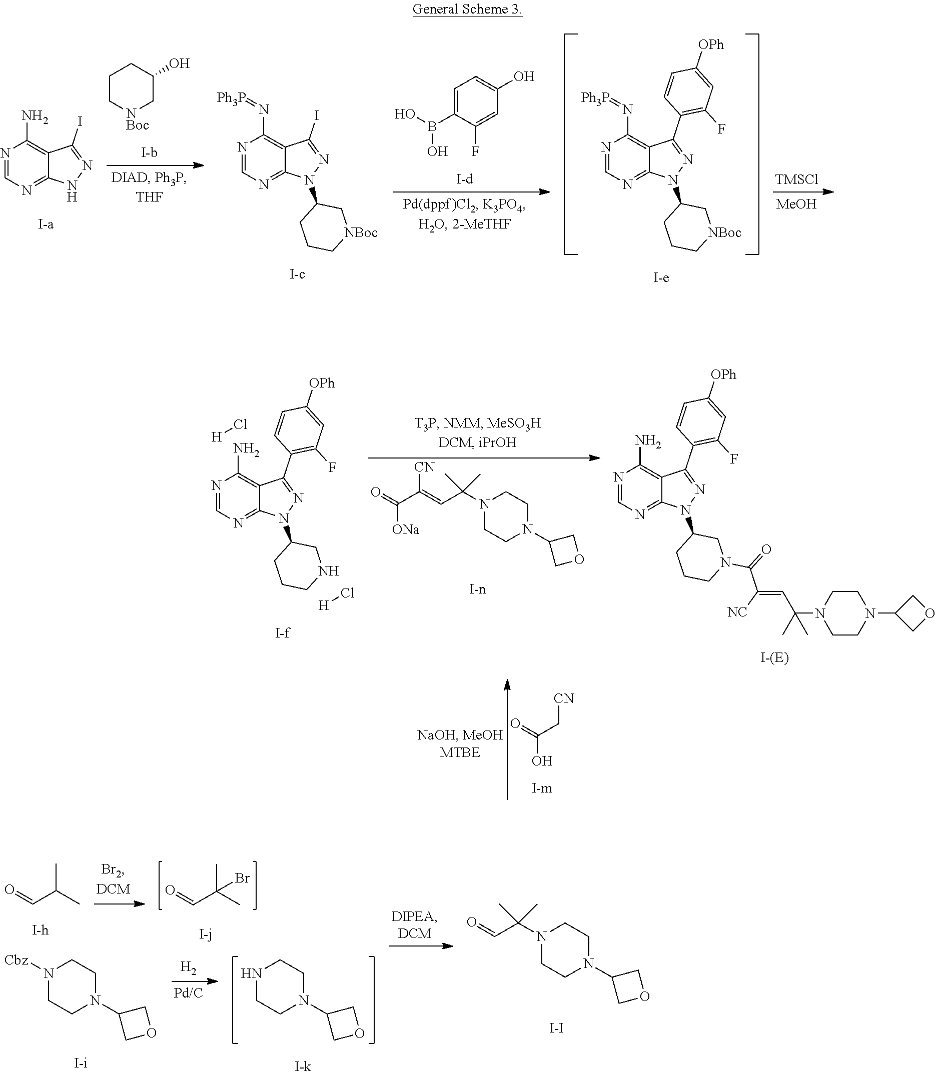

### Example S1. Synthesis of Compound I-g

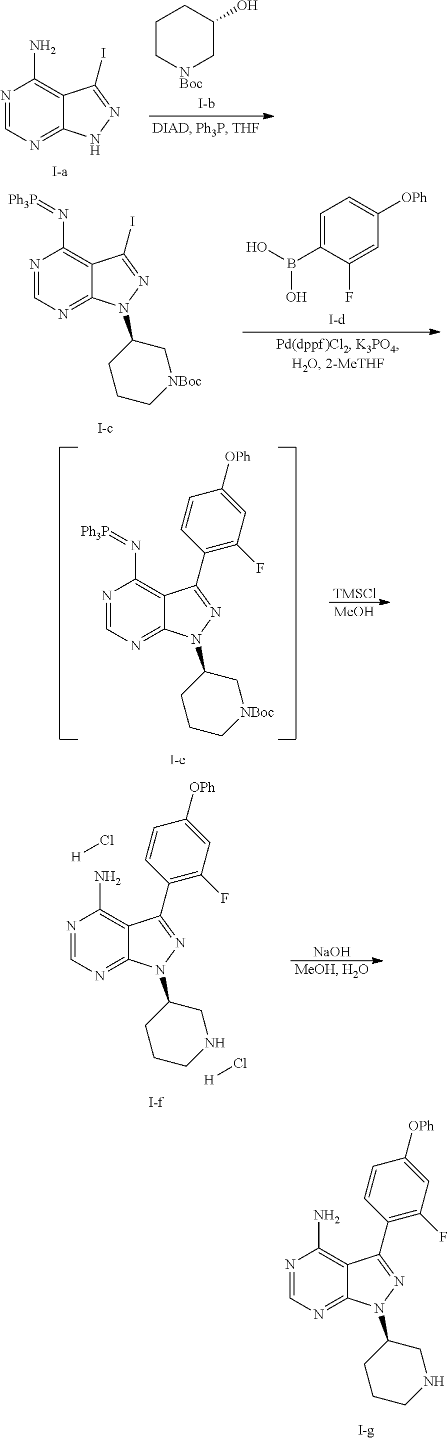

### Example S1.1 Compound I-c

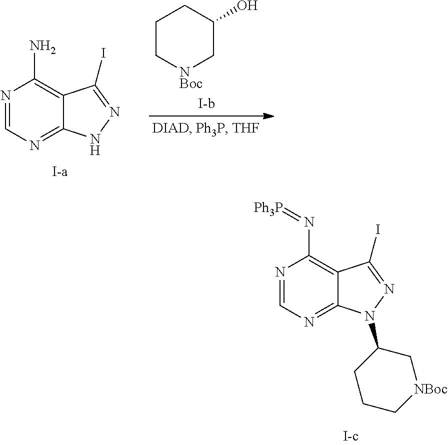

Compound I-a (20.26 g), triphenylphosphine (70.4 g, 3.5 eq) and Compound I-b (30.58 g, 2.00 eq) were charged in a reactor and THF was added (405 mL). The mixture was stirred while diisopropyl azodicarboxylate (54 g, 3.46 eq) was dosed at 0° C. over 5 hours. After complete conversion of compound I-a, the reaction was quenched by adding water (7 mL, 5 eq). The reaction mixture was concentrated to 250 mL and a solvent swap was performed from THE to methanol. Compound I-c was filtered, to which was added methanol (126 mL) to re-form a slurry. Compound I-c was filtered and dried under vacuum to obtain 40.9 g (73% yield).

### Example S1.2 Compound I-f

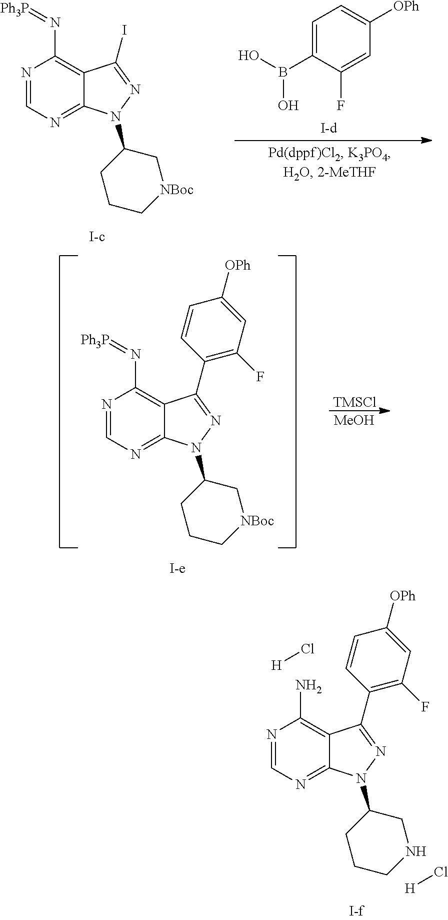

Compound I-d (19.4 g, 1.21 eq), compound I-c (49.6 g, 1.00 eq), K₃PO₄ (37.6 g, 2.59 eq), and Pd(dppf)Cl₂ (0.48 g, 0.01 eq) were charged into a reactor. 2-MeTHF (397 mL) and water (96 mL) were added. The reaction mixture was heated to 73° C. After 20 hours, the reaction was cooled to 20° C. and diluted with 2-MeTHF (195 mL) and water (178 mL). After phase separation, the organic phase was washed with 5% sodium chloride solution (178 g). Half of the aqueous layer was removed, and the solution was filtered on charcoal. After phase separation of the filtrate, the remaining aqueous phase was discarded. The organic phase was washed with 20% aqueous sodium chloride solution (204 g) and diluted with methanol (139 mL). Trimethylsilyl chloride (45.5 g, 6.06 eq) was added at 20° C. over 4 h. The reaction mixture was heated to 42° C. and was stirred for 19 h. Water (26 mL) was added to the reaction mixture, which was heated to 65° C. for 3 h. The mixture was then slowly cooled down to 25° C. and 2-MeTHF was added. The solid obtained was filtered at 0° C., washed with 2-MeTHF (384 mL), and dried under vacuum at 80° C. to obtain 29.9 g (91% yield) of compound I-f. As described in Example S1.3, isolated compound I-f can be used to create compound I-g. However, it is not necessary to isolate compound I-f. Instead, compound I-f can be created in situ and used directly to create compound I-g. See Example S1.4 below.

### Example S1.3 Compound I-g

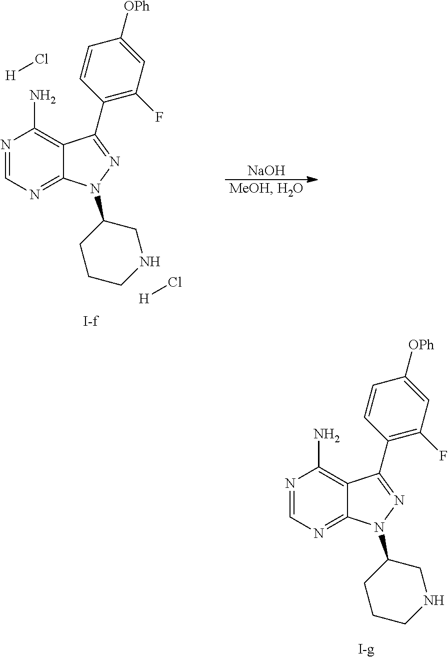

Compound I-f (100 g, 1.00 eq) was solubilized in methanol (200 mL). The solution was filtered on charcoal. Water (336 mL) was added and the solution was heated to 50° C. The pH was adjusted to 7.3 by addition of an 30% aqueous sodium hydroxide solution. The mixture was seeded with compound I-g and the pH was adjusted to 12 by adding 30% aqueous sodium hydroxide solution. The suspension was diluted with water (100 mL) and the temperature was cooled down to 20° C. The solid was filtered, washed with a mixture of water/methanol (70/30), and dried at 40° C. under vacuum. 83.9 g (99% yield) of compound I-g was obtained. The compound I-g seed was obtained following the same synthesis steps described, but without the seeds and using a 45% aqueous sodium hydroxide solution. Spontaneous crystallization was observed after the cooling step to 20° C. Keeping the residual triphenylphosphine oxide to less than 1% was crucial for successful crystallization.

¹H NMR (400 MHz, DMSO-d6) δ ppm: 1.65 (m, 1H) and 1.88 (dquintuplet, J=14 Hz, 3.5 Hz, 3.5 Hz, 3.5 Hz, 3.5 Hz, 1H), 2.18 (m, 1H), 2.29 (qd, J=12 Hz, 12 Hz, 12 Hz and 4 Hz), 2.73 (ddd, J=13−12 and 3 Hz, 1H), 3.05 (dt, J=13 Hz and 3.3 Hz, 1H), 3.27 (m, 2H), 4.82 (m, 1H), 5.8 (br, NH₂), 6.86 (dd, J=11 & J=2.5 Hz, 1H), 6.94 (dd, J=8.5 & J=2.5 Hz, 1H), 7.10 (dd, J=7.5 & J=1 Hz, 1H), 7.21 (t, J=7.5 Hz, 1H), 7.41 (dd, J=8 7.5 Hz, 1H), 7.53 (t, J=8.5 Hz, 1H), 8.33 (s, 1H).

$${MS [ M + H ]}^{+} = 405.2 .$$

### Example S1.4 Compound I-g

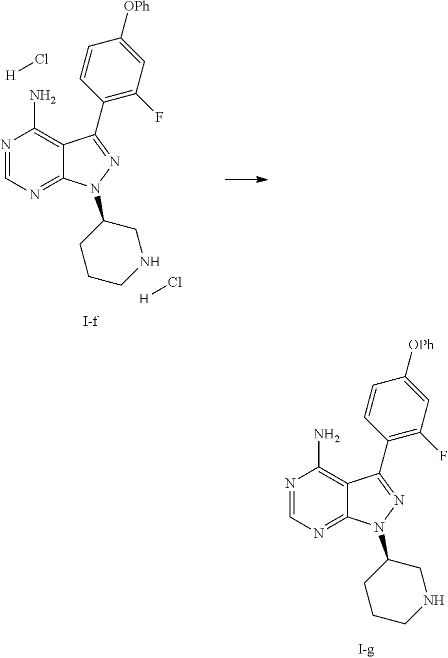

Compound I-f was prepared according to Example S1.2 above. However, rather than isolating the compound as a solid, the 2-MeTHE solution containing compound I-f (mass of about 115 g or 4.12 g of compound I-f) was liquid-liquid phase extracted twice with ˜50 mL and ˜30 mL water under stirring and at 20°±3° C. Aqueous phases were combined and treated twice with 10 mL organic toluene. About 97% of initial TPPO content was removed during the phase extractions. Aqueous phase containing compound I-f was basified with NaOH solution (23%) at 20°±3° C. under stirring, until reach a pH about 12.5 (8.4 g NaOH 23% added). Two phases were then observed, an oiling orange aqueous phase and an organic white and cloudy phase. Liquid-liquid separation using 8 mL DCM was performed three time to give three mixed organic phases.

After separation of organic an aqueous phase, a swap solvent DCM/MeOH was carried out on organic phase to remove dichloromethane. 16 mL H₂O was gradually added at 50° C. Following precipitation of mixture, the suspension was cooled at 20° C. and stirred at 200 rpm for 12 h. The precipitation was isolated by filtering (5.32 g of wet cake) and then vacuum dried (10 mbars) during 72 h at 25° C. to give 3.59 g compound I-g as dried solid.

### Example S2. Synthesis of Compound I-n

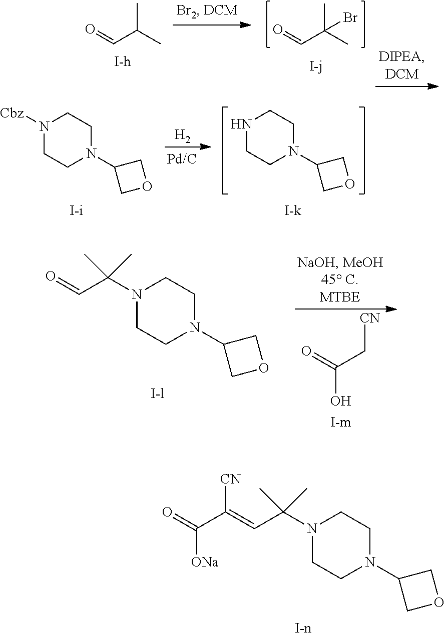

### Example S2.1. Compound I-l

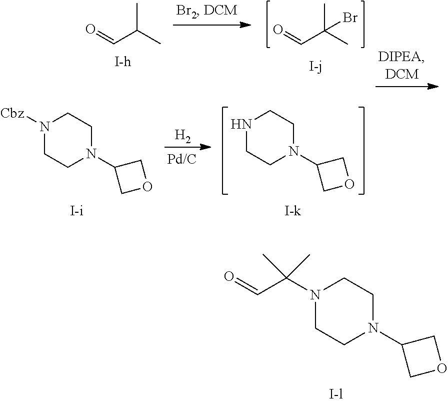

Compound I-l was prepared as described in Owens et al. J. Med. Chem., 2022, 65, 7, 5300-5316, WO2014/039899A1, and WO2014/089379A1, or via commonly known chemical reactions.

### Example S2.2. Compound I-n

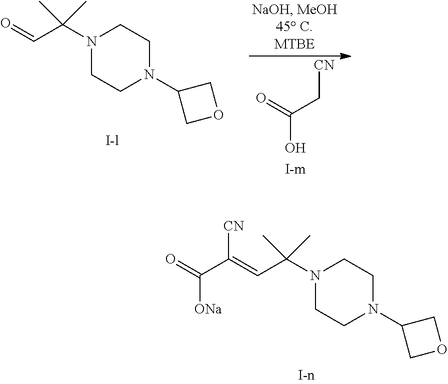

Compound I-m (123.6 g, 1.00 eq) and compound I-I (340.9 g, 1.10 eq) were solubilized in methanol (2000 mL). The solution was heated to 50° C. Sodium hydroxide pellets (115.5 g, 2.00 eq) were added portion-wise. The reaction was stirred at 50° C. for 2 h. MTBE (4000 mL) was added. The resulting suspension was cooled down to 5° C. and filtered. The solid was washed with a mixture of MTBE/MeOH (2/1) and with MTBE. 396 g (92% yield) of compound I-n was obtained.

¹H NMR (400 MHz, DMSO-d6) δ ppm: 1.24 (s, 6H), 2.49 (s, 4H), 2.27 (s, 4H), 3.36 (quintuplet, J=6.5 Hz, 1H), 4.40-4.50 (t, J=6.5 & 6.0 Hz, 4H), 7.15 (s, 1H).

MS [M+H]⁺=280.

### Example S3. Synthesis of Compound of Formula I-(E)

### Example S3.1 Synthesis of the Compound of Formula (I-(E)) from Compound I-f

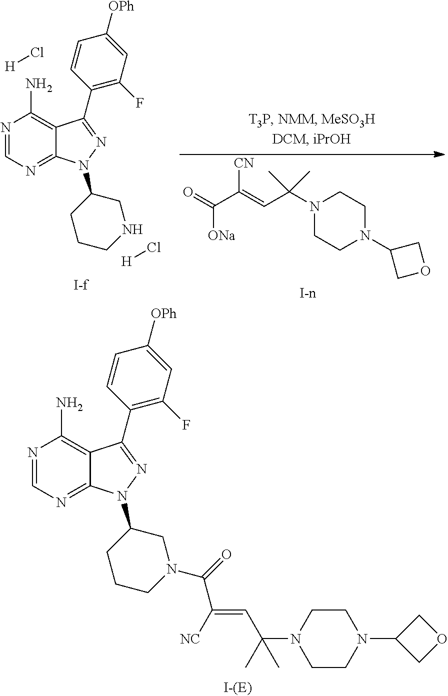

Compound I-f (21.2 g) was suspended in dichloromethane (235 mL). N-methyl-morpholine (37.4 g, 6.0 eq), and propan-2-ol (12.5 mL) were added. The solution was heated to 30° C. Compound I-n (23.28 g, 1.25 eq) was added and T₃P in dichloromethane (59 g, 1.50 eq) was dosed over 30 min. After the reaction was complete, the reaction mixture was washed with aqueous sodium chloride solution. Water was added to the organic phase and the pH was adjusted to 4.5 with methanesulfonic acid. After phase separation, methanesulfonic acid (24 g) was added to the organic phase and the solution was stirred for at least 3 h at 30° C. After cooling to 10° C., water was added. After phase separation, dichloromethane was added to the aqueous phase then the pH was adjusted to 3.2-3.5 with 10% solution of potassium hydroxide. After decantation, the combined organic phase was washed with water. Then water was added to the organic phase and the pH was adjusted to 7.5 with a potassium hydroxide aqueous solution at 1%. After phase separation, 36.46 g (89% yield) of the compound of Formula (I-(E)) was obtained in a dichloromethane solution.

### Example S3.2 Synthesis of the Compound of Formula (I-(E)) from Compound I-g

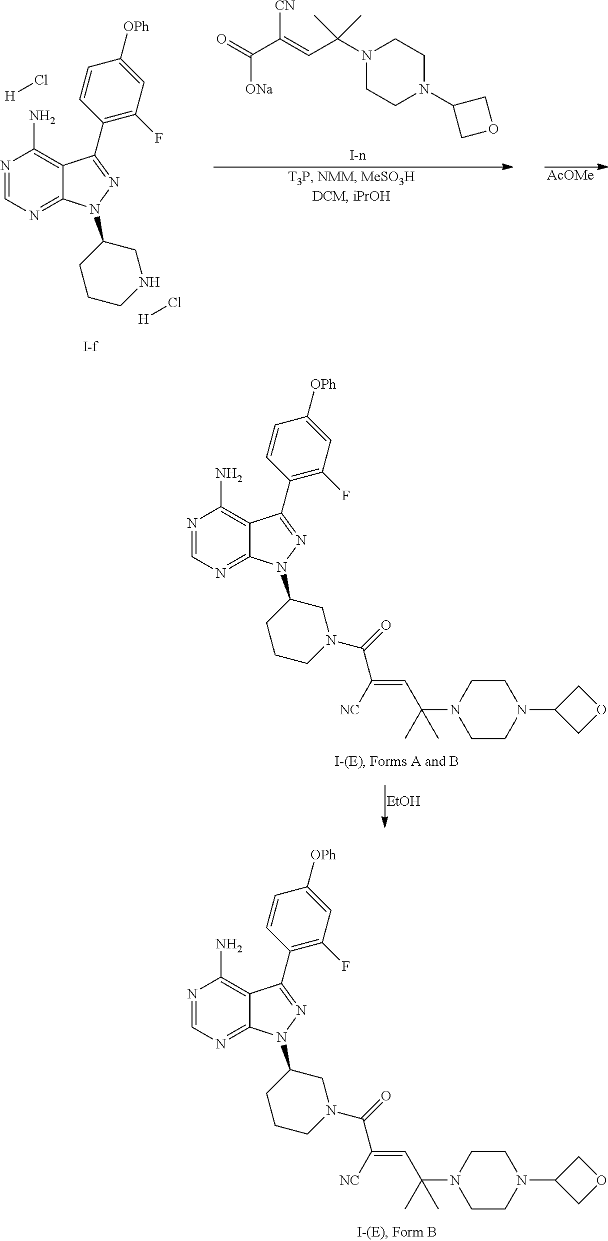

Compound I-g (25 g) was solubilized in dichloromethane (235 mL). N-methyl-morpholine (31.2 g, 5.0 eq), methanesulfonic acid (5.94 g, 1 eq), and propan-2-ol (12.5 mL) were added. The solution was heated to 30° C. Compound I-n (23.28 g, 1.25 eq) was added and T₃P in dichloromethane (59 g, 1.50 eq) was dosed over 30 min. After the reaction was complete, the reaction mixture was washed with aqueous sodium chloride solution. Water was added to the organic phase and the pH was adjusted to 4.5 with methanesulfonic acid. After phase separation, methanesulfonic acid (24 g) was added to the organic phase and the solution was stirred for at least 3 h at 30° C. After cooling to 10° C., water was added. After phase separation, dichloromethane was added to the aqueous phase then the pH was adjusted to 3.2-3.5 with 10% solution of potassium hydroxide. After decantation, the combined organic phase was washed with water. Then water was added to the organic phase and the pH was adjusted to 7.5 with a potassium hydroxide aqueous solution at 1%. After phase separation, 36.46 g (89% yield) of the compound of Formula (I-(E)) was obtained in a dichloromethane solution.

As an alternative, after adding the solution of the compound of Formula (I-(E)) in DCM into a double jacket reactor, the DCM organic phase was concentrated under vacuum from 7.7 UO to 3.3 UO under a pressure of 150 mbar.

### Example S4. Preparation of a Mixture of Crystalline Form A/Form B of the Compound of Formula (I-(E)) through Crystallization of the Methylacetate Solvate of the Compound of Formula (I-(E))

The solution of the compound of Formula (I-(E)) in dichloromethane was solvent exchanged to a methylacetate solution. The compound of Formula (I-(E)) was then crystallized in 4% w/w DCM in methylacetate (total solvent 5.7 UO). This crystallization solution was seeded with wet methylacetate solvate of the compound of Formula (I-(E)) (0.5% to 1% w/w) at about 20° C. The methylacetate solvate seeds of the compound of Formula (I-(E)) were obtained according to methods as described in Example S7.2.1. The suspension was then cooled down and maintained at 0° C. (−10° C./h). The suspension was then filtrated and washed with methylacetate before performing filtration and drying, which gave wet methylacetate solvate of the compound of Formula (I-(E)). The crystallization yield was about 90%. Drying the wet methylacetate solvate of the compound of Formula (I-(E)) led to its conversion into a mixture of crystalline Form A and crystalline Form B of the compound of Formula (I-(E)). The ratio of crystalline Form A/Form B differed based on the drying conditions. Drying was carried out to minimize the ratio of crystalline Form A over crystalline Form B of the compound of Formula (I-(E)), which was performed under nitrogen flow and under vacuum at a pressure range between 50 and 600 mbars at a temperature between 0° C. and 50° C.

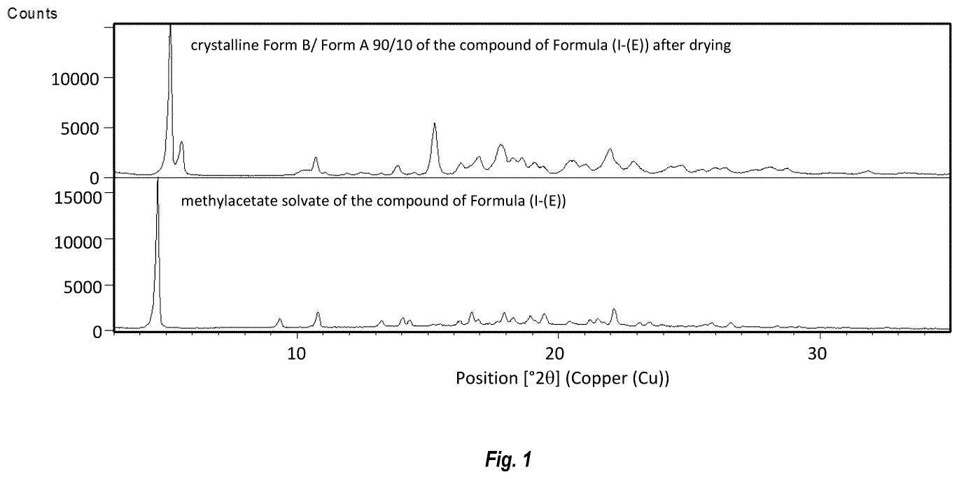

The XRPD patterns of a methylacetate solvate of the compound of Formula (I-(E)) and a mixture of crystalline Form B/Form A 90/10 of the compound of Formula (I-(E)) after drying are shown in FIG. 1.

### Example S5. Preparation of Crystalline Form B of the Compound of Formula (I-(E)) by Polishing

### Example S5.1 Polishing Step Using Ethanol

A process for polishing the compound of Formula (I-(E)) as made as set forth herein is partially dissolving the compound of Formula (I-(E)) mixture of crystalline Form A and crystalline Form B in 5 volumes of absolute ethanol and utilizing their different solubilities in ethanol: crystalline Form A exhibits a higher solubility in ethanol than crystalline Form B of the compound of Formula (I-(E)). For example, the suspension of the compound of Formula (I-(E)) in ethanol was heated up to 30-43° C. Several cycles of heating/cooling were applied until crystalline Form A completely dissolved in ethanol, which was monitored by XRPD analysis. Once only crystalline Form B was observed in the suspension, the mixture was cooled to 0° C. (cooling rate: −10° C./h) to crystallize the compound of Formula (I-(E)) into crystalline Form B. After drying, crystalline Form B of the compound of Formula (I-(E)) was obtained with a >90% yield.

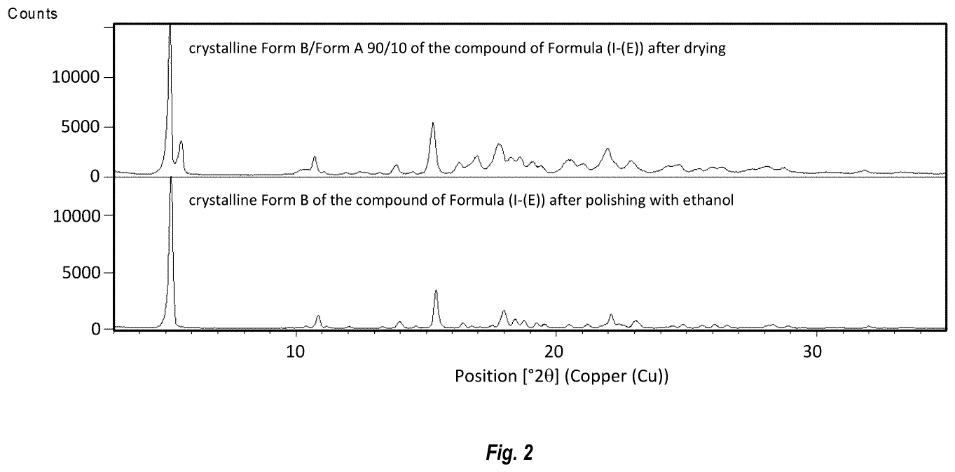

The XRPD patterns of a mixture of crystalline Form B and crystalline Form A 90/10 of the compound of Formula (I-(E)) after drying and crystalline Form B of the compound of Formula (I-(E)) after the polishing step with ethanol are shown in FIG. 2.

Although the present disclosure has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

### Example S5.2 Polishing Step Using Isopropylacetate

0.1368 g of a mixture of crystalline Form A and crystalline Form B of the compound of Formula (I-(E)) was partially dissolved in 1.2993 g of IPAC. The suspension was heated stepwise to 30° C., 40° C., 50° C., and finally 60° C. in a double jacketed reactor for 2 h total. The suspension was then cooled down to RT within 2 h and magnetically stirred for 12 h.

65.8 mg (Y=48%) of an off-white, pale yellowish solid of structurally pure crystalline Form B was obtained after filtering and drying overnight at RT.

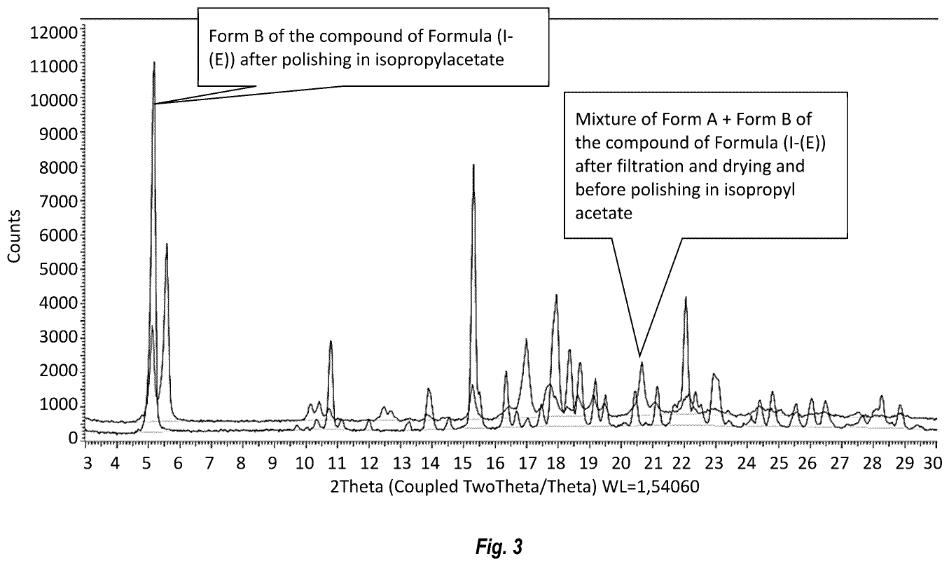

The XRPD patterns of crystalline Form B of the compound of Formula (I-(E)) obtained after the polishing step with IPAC is shown in FIG. 3.

### Example S6. Preparation of Crystalline Form B Using Pressure

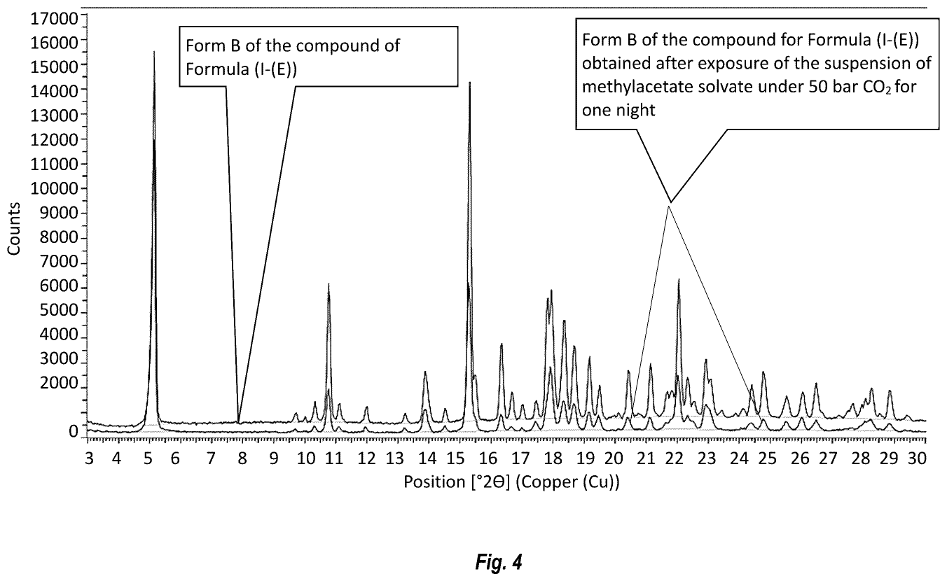

A suspension of the methylacetate solvate of the compound of Formula (I-(E)) (without any trace of crystalline Form A and/or Form B) was placed overnight in a reactor under 50 bars of CO₂. Only crystalline Form B of the compound of Formula (I-(E)) was observed, even without the polishing steps described above, with a 100% yield (FIG. 4).

### Example S7. Additional Preparation and Characterization of the Compound of Formula (I-(E)) Solvates

### Example S7.1. Analytical Methods

### X-Ray Powder Diffraction (XRPD)

XRPD measurements were carried out at ambient temperature on a Bruker D8 Discover apparatus using the following conditions: Cu Kα radiation (1.54 Å), Ni Kβ filter, divergent slit 0.6 mm, and high-performance detector (Lynx-eye).

The samples were scanned from 3 to 30° 2θ angles, with a 0.04° step (1 s duration per step). 40 kV-40 mA.

The software used to process the data was Bruker DIFFRAC.EVA V4.1.

### Optical Microcopy and HT-Microscopy

Nikon Eclipse LV100 optical binocular microscope equipped with a Nikon Digital sight DS-Ri1 camera was used for microscopy studies.

The software used to analyze the results was Nikon NIS Elements D V3.1.

To perform temperature-regulated microscopy, a hot/cold stage chamber Linkam THMS 600 was used (temperature range from −196 to +600° C.). Heating and cooling were controlled with a Peltier system associated to the circulation of liquid nitrogen that can provide sub-ambient temperatures down to −196° C.

The device was controlled with the software Linksys 32 v2.4.3.

### Differential Scanning Calorimetry (DSC)

DSC measurements were performed on a Netzsch DSC 214 Polyma apparatus equipped with an intra-cooler and purged by nitrogen. The device was controlled with the software Netzsch Proteus Measurements v7.1.0.

Samples were weighed in 25 μL aluminum pans.

The software used to exploit the results was Netzsch Proteus Thermal Analysis v7.1.0.

### Differential Scanning Calorimetry (DSC)—Thermogravimetric Analysis (TGA)

The following conditions were used for DSC-TGA measurements.
Netzsch STA 449C
Aluminum pan, pierced lid
Atmosphere: He
Heating rate=5K·min⁻¹
Data acquisition and processing: Netzsch Proteus Thermal Analysis software (v. 6.1.)

### Example S7.2. Analysis Results of the Compound of Formula (I-(E)) Solvates

### Example S7.2.1. Preparation of the Methylacetate Solvate of the Compound of Formula (I-(E))

The methylacetate solvate of the compound of Formula (I-(E)) was obtained from the acetonitrile solvate of the compound of Formula (I-(E)) (or crystalline Form C of the compound of Formula (I-(E))) or the crystalline Form B of the compound of Formula (I-(E)).

### Methylacetate Solvate of the Compound of Formula (I-(E)) from Crystalline Form C

10.35 g of the acetonitrile solvate of the compound of Formula (I-(E)) (or crystalline Form C of the compound of Formula (I-(E))) was completely dissolved in 31 g of methylacetate (MAC) at RT. Dissolution was fast and the medium remained turbid. After 20 minutes, crystallization of methylacetate solvate of the compound of Formula (I-(E)) was progressing. After 4 h, the white slurry was thick.

### Methylacetate Solvate of the Compound of Formula (I-(E)) from Crystalline Form B

Crystalline Form B of the compound of Formula (I-(E)) was dissolved in 5V of methylacetate. The mixture was heated at 40° C. and maintained at 40° C. for 1 h. The mixture was cooled at between 0 and 10° C.

### Methylacetate Solvate of the Compound of Formula (I-(E)) from Amorphous Form

32.82 g of the amorphous form of the compound of Formula (I-(E)) was dissolved into 60 mL of methyl acetate at RT. The solution was seeded with 150 mg of crystalline Form B of the compound of Formula (I-(E)). The suspension was maintained at RT for 24 h under magnetic stirring. The methylacetate solvate of the compound of Formula (I-(E)) was obtained.

### Example S7.2.2. Characterization of the Methylacetate Solvate of the Compound of Formula (I-(E))

The crystalline structure of the methylacetate solvate of the compound of Formula (I-(E)) was analyzed and the crystalline parameters were measured.

### Crystalline Structure

The crystalline structure of the methylacetate solvate of the compound of Formula (I-(E)) was obtained. The data recording was performed at a low temperature of 130 K.

The ordered structure was refined with high resolution (4.4%) and showed the presence of one molecule of solvent (methylacetate) per one compound of Formula (I-(E)) molecule. The methylacetate solvate of the compound of Formula (I-(E)) was considered to be a monosolvate.

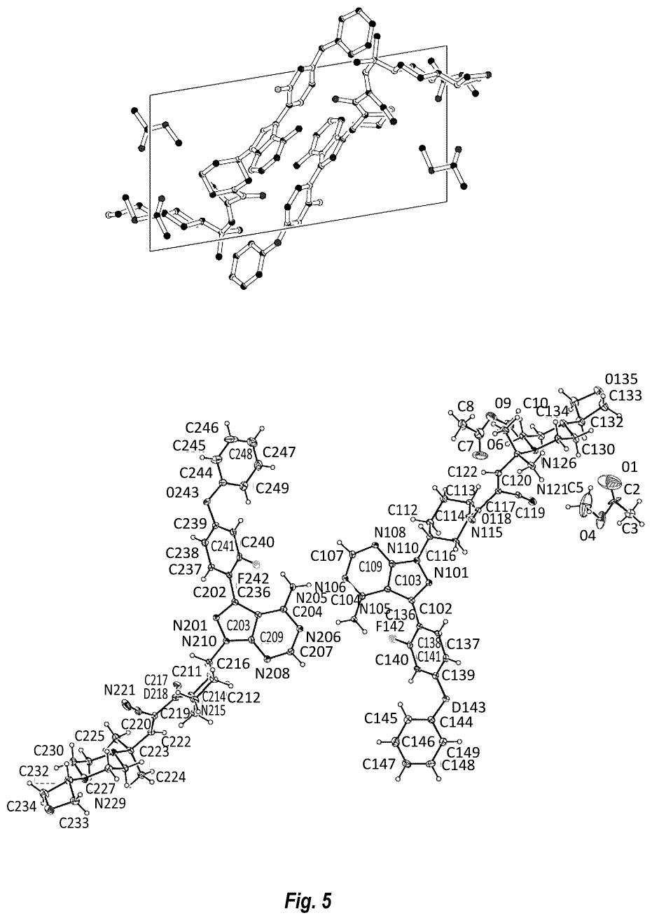

The crystalline structure of the methylacetate solvate of the compound of Formula (I-(E)) is shown in FIG. 5.

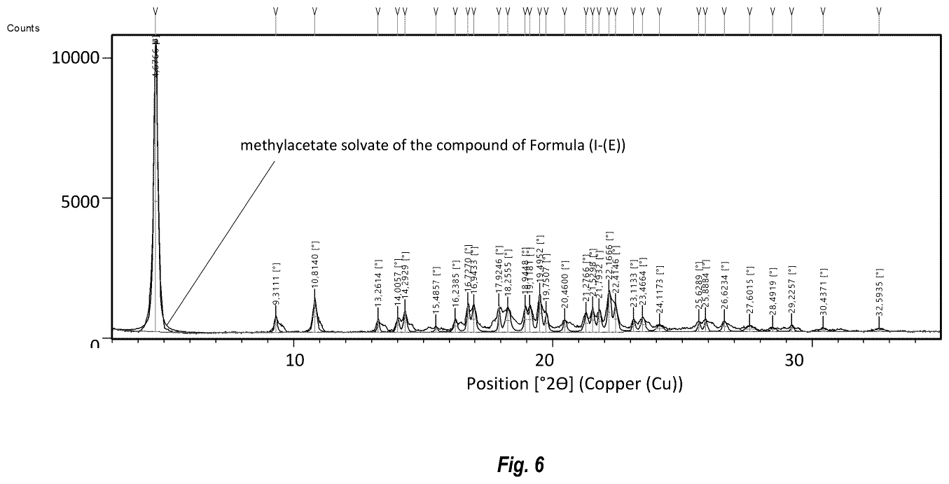

The XRPD pattern was measured on Bruker D8 advance diffractometer following the method described above, which is shown in FIG. 6.

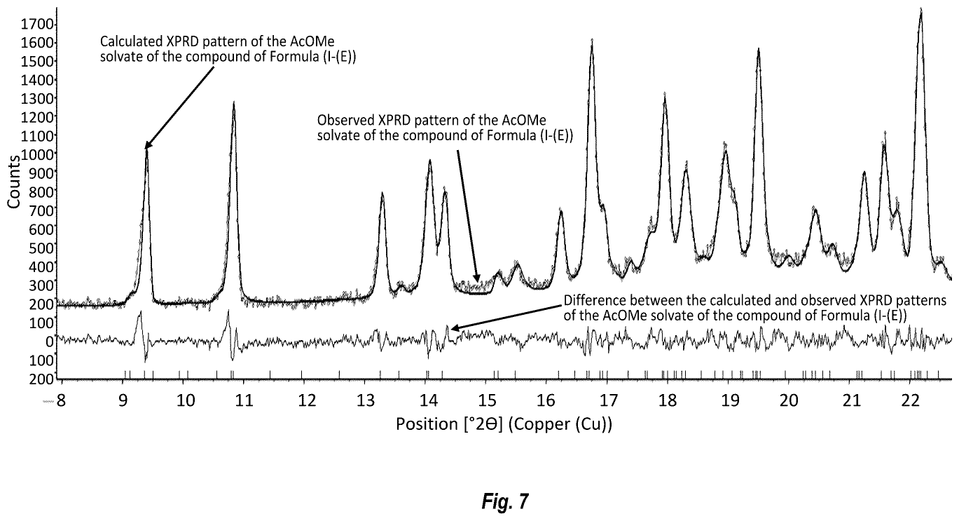

The XPRD pattern of the AcOMe solvate of the compound of Formula (I-(E)) after Pawley refinement is shown in FIG. 7.

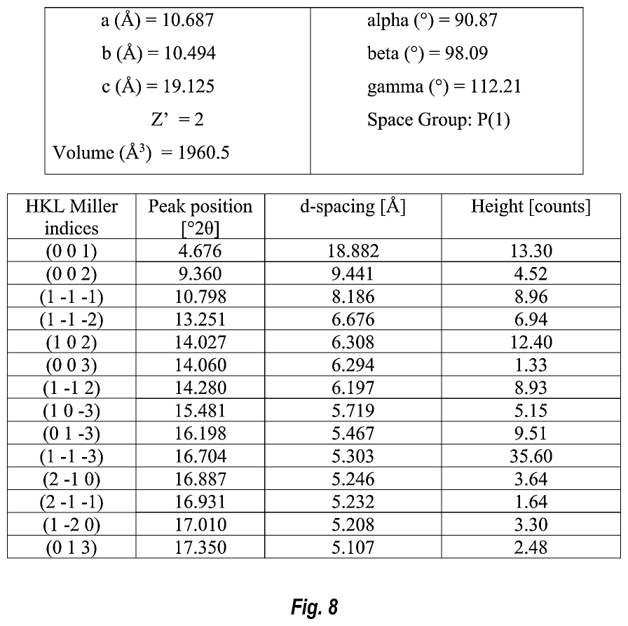

The unit cell parameters for the AcOMe solvate of the compound of Formula (I-(E)) is shown in in FIG. 8.

### Microscopy Observations

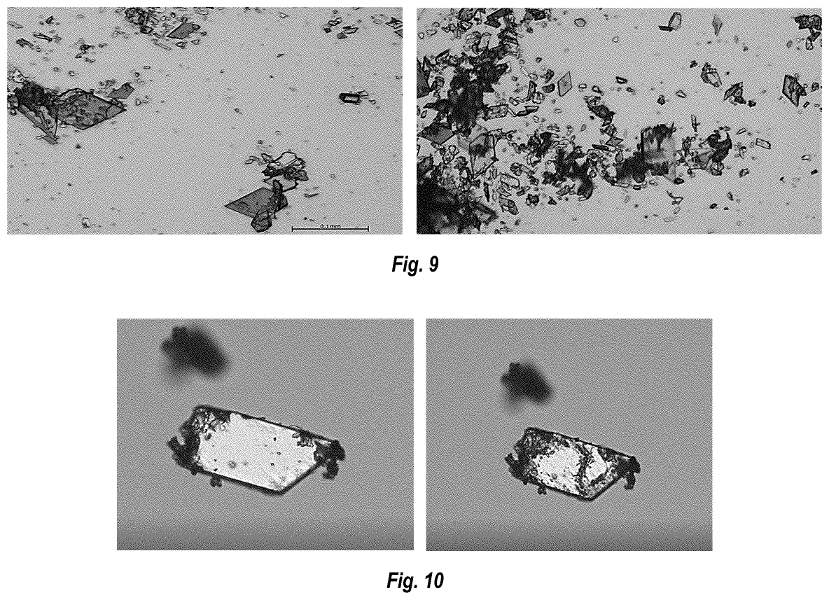

The methylacetate solvate of the compound of Formula (I-(E)) was analyzed by polarized optical microscopy. Crystals of methylacetate solvate of the compound of Formula (I-(E)) were observed as a thin platelet habitus as shown in FIG. 9.

Defects such as micro cracks on the crystal surface were observed after the desolvation process as shown in FIG. 10 (left: solvate crystal before desolvation, right: solvate crystal after desolvation with micro-crack (default)). This desolvation phenomenon was analyzed using hot stage microscopy according to the method described above.

### Example S7.2.3. Preparation of the Dimethylcarbonate Solvate of the Compound of Formula (I-(E))

Dimethylcarbonate solvate of the compound of Formula (I-(E)) was also an intermediate in the synthesis of crystalline Form B of the compound of Formula (I-(E)). This material was obtained from the acetonitrile solvate of the compound of Formula (I-(E)), the crystalline Form B of the compound of Formula (I-(E)), or the amorphous form of the compound of Formula (I-(E)).

### Dimethylcarbonate Solvate of the Compound of Formula (I-(E)) from Crystalline Form C of the Compound of Formula (I-(E))

0.7191 g of crystalline Form C of the compound of Formula (I-(E)) was completely dissolved in 5.0961 g of dimethylcarbonate. The solution was placed in a freezer for several hours until the completion of solidification. The vial was then placed in RT. After melting of the solvent, a foggy medium was obtained. This light suspension was stirred for hours at RT to give a white thick suspension. The particles were fine enough so that even after several days without magnetic stirring, there was no sedimentation at RT.

### Dimethylcarbonate Solvate of the Compound of Formula (I-(E)) from Amorphous Form of the Compound of Formula (I-(E))

7 g of amorphous form of the compound of Formula (I-(E)) was dissolved in 21 mL of dimethylcarbonate solvent. The solution was seeded with few milligrams of crystalline Form B of the compound of Formula (I-(E)) and frozen at −18° C. After 12 h, the mixture was thawed and stirred at RT and crystallization was observed. The solid obtained was dimethylcarbonate solvate of the compound of Formula (I-(E)).

### XRPD Analysis of the Dimethylcarbonate Solvate of the Compound of Formula (I-(E))

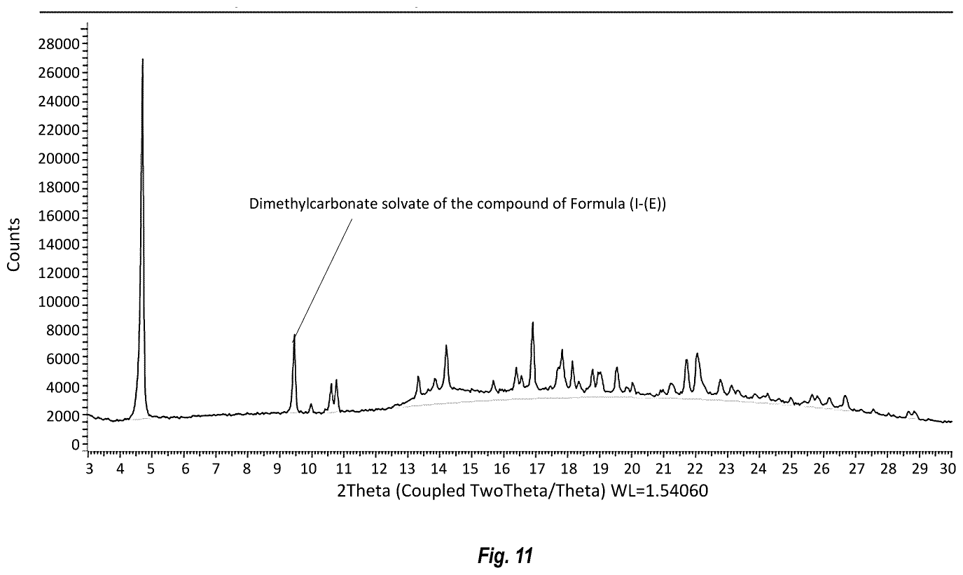

The dimethylcarbonate solvate of the compound of Formula (I-(E)) was analyzed by XRPD following the method described above. The pattern recorded and peaks positions are presented in the FIG. 11.

Based on peak positions, the XRPD pattern of the dimethylcarbonate solvate of the compound of Formula (I-(E)) was comparable to that of the methylacetate solvate of the compound of Formula (I-(E)). The 2 pseudopolymorphs were likely to be structurally very close (possibly isomorphous).

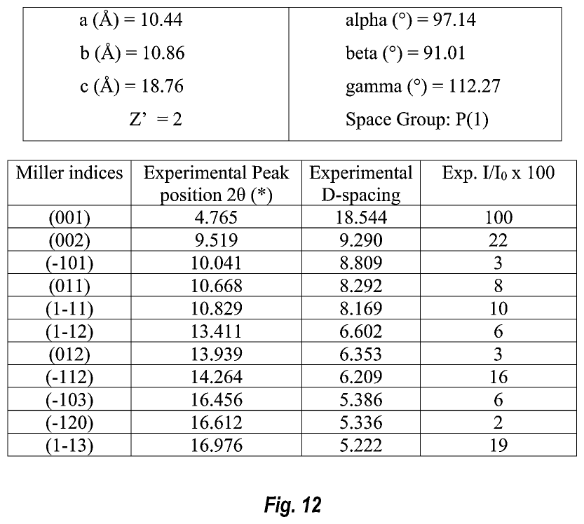

The unit cell parameters for the DMC solvate of the compound of Formula (I-(E)) in FIG. 12.

### Thermal Analysis of the Dimethylcarbonate Solvate of the Compound of Formula (I-(E))

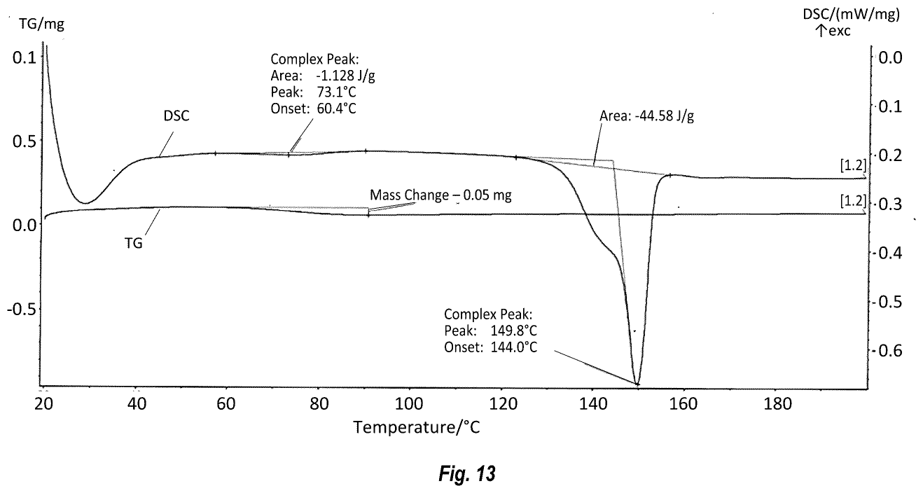

The dimethylcarbonate solvate of the compound of Formula (I-(E)) was analyzed by calorimetry. Thermograms obtained are presented in FIG. 13.

Results indicated facile desolvation of the dimethylcarbonate solvate of the compound of Formula (I-(E)) from ambient temperature to 90° C. This desolvation seemed to lead to a mixture of crystalline Form A and crystalline Form B of the compound of Formula (I-(E)), based on the endothermic event recorded from about 120° C. to 155° C. The ratio of crystalline Form A/Form B of the compound of Formula (I-(E)) varied depending on desolvation conditions.

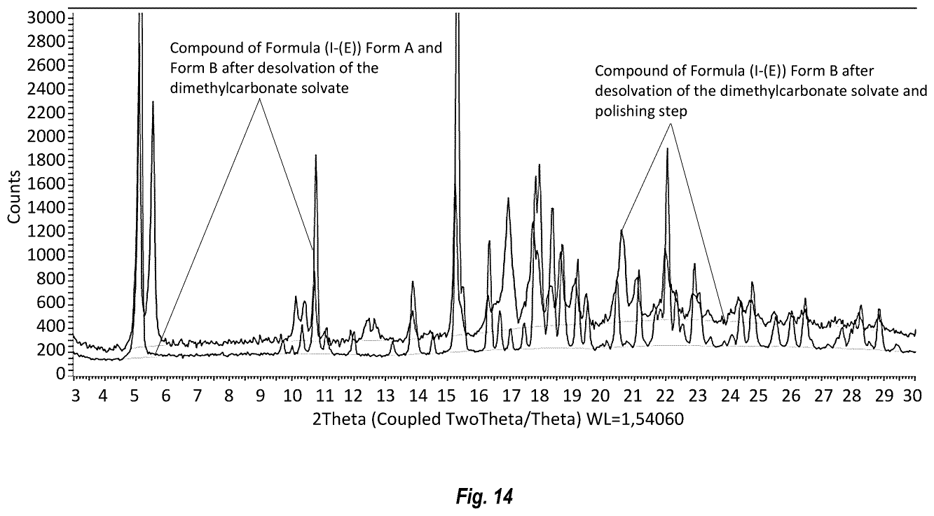

To confirm that desolvation indeed leads to a mixture of crystalline Form A and crystalline Form B, dimethylcarbonate solvate of the compound of Formula (I-(E)) was held under 100° C. for 4 h and an X ray analysis was performed. Results showed the desolvation of the dimethylcarbonate solvate resulted in a mixture of crystalline Form A and crystalline Form B as shown in FIG. 14.

### Microscopy Observations

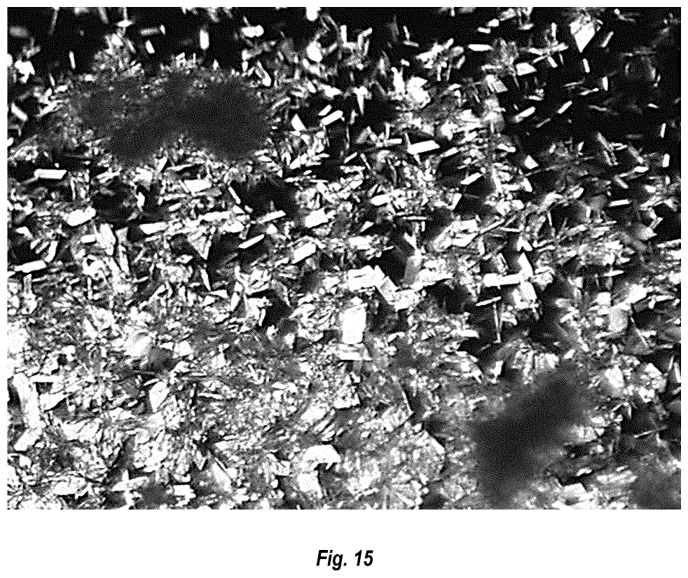

Crystals of the dimethylcarbonate solvate of the compound of Formula (I-(E)) were analyzed by polarized optical microscopy as shown in FIG. 15. The crystals appeared as thin platelet habitus.

Although the present disclosure has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

### Embodiments

Non-limiting embodiments of the disclosure include:

Embodiment 1. A method of preparing a compound of Formula (I):

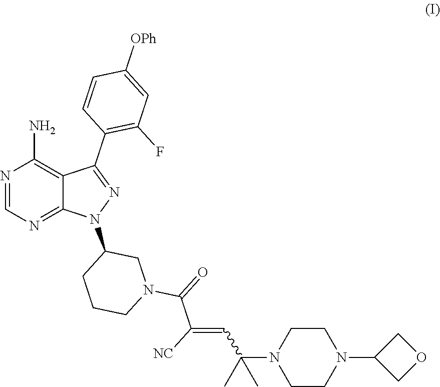 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

or a salt thereof, comprising:
reacting a compound of Formula (I-g):

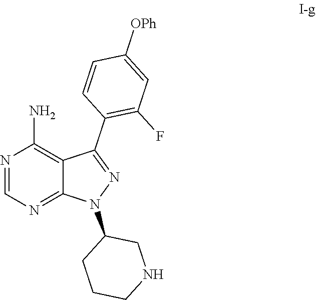 [SMILES: Nc1ncnc2c1c(-c1ccc(Oc3ccccc3)cc1F)nn2[C@@H]1CCCNC1]

or a salt thereof, with a compound of Formula (I-n):

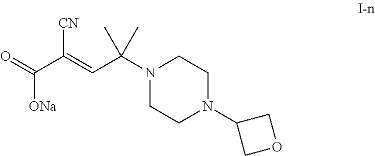

or a salt thereof, with amide-coupling reagent, organic base, and acid in organic solvent to form the compound of Formula (I).

Embodiment 2. The method of embodiment 1, wherein the amide-coupling reagent is propylphosphonic anhydride.

Embodiment 3. The method of embodiment 1, wherein the organic base is N-methylmorpholine, triethylamine, or diisopropylamine.

Embodiment 4. The method of embodiment 1, wherein the acid is hydrochloric acid, methanesulfonic acid, paratoluenesulfonic acid, or chlorotoluenesulfonic acid.

Embodiment 5. The method of embodiment 1, wherein the organic solvent comprises dichloromethane, dichloroethane, acetonitrile, chloroform, 2-methyltetrahydrofuran, tetrahydrofuran, methyl tert-butyl ether, toluene, chlorobenzene, methanol, ethanol, or isopropanol.

Embodiment 6. The method of embodiment 1, wherein the organic solvent comprises dichloromethane and isopropanol.

Embodiment 7. The method of any one of embodiments 1-6, wherein the compound of Formula (I-n), or a salt thereof, is prepared by reacting a compound of Formula (I-l):

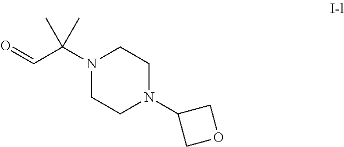

or a salt thereof, with a compound of Formula (I-m):

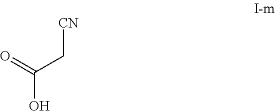

or a salt thereof, with inorganic or organic base in organic solvent to form the compound of Formula (I-n).

Embodiment 8. The method of embodiment 7, wherein the inorganic base is sodium hydroxide, lithium hydroxide, or potassium hydroxide.

Embodiment 9. The method of embodiment 7, wherein the organic base is sodium methoxide.

Embodiment 10. The method of embodiment 7, wherein the organic solvent is alcohol.

Embodiment 11. The method of embodiment 10, wherein the alcohol is methanol.

Embodiment 12. The method of any one of embodiments 1-6, wherein the compound of Formula (I-g), or a salt thereof, is prepared by reacting a compound of Formula (I-f):

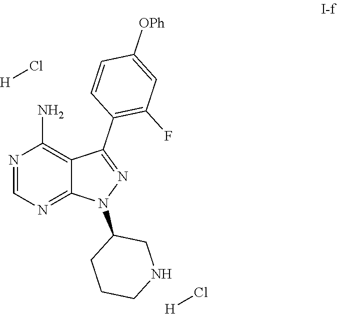

or a salt thereof, with base in alcohol and water to form the compound of Formula (I-g).

Embodiment 13. The method of embodiment 12, wherein the base is sodium hydroxide.

Embodiment 14. The method of embodiment 12, wherein the alcohol is methanol.

Embodiment 15. The method of any one of embodiments 1-14, wherein the compound of Formula (I), or a salt thereof, is at least 95% by weight a compound of Formula (I-(E)):

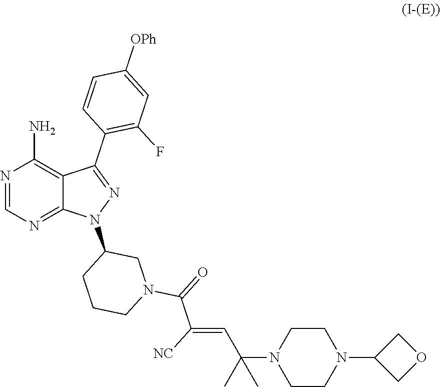 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

or a salt thereof.

Embodiment 16. A method of preparing a compound of Formula (I-n):

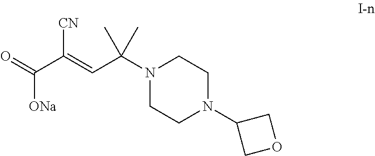

or a salt thereof, by reacting a compound of Formula (I-l):

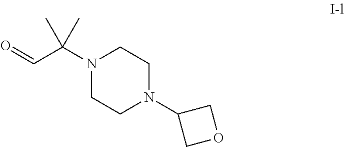

or a salt thereof, with a compound of Formula (I-m):

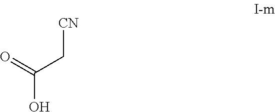

or a salt thereof, with inorganic or organic base in organic solvent to form the compound of Formula (I-n).

Embodiment 17. The method of embodiment 16, wherein the inorganic base is sodium hydroxide, lithium hydroxide, or potassium hydroxide.

Embodiment 18. The method of embodiment 16, wherein the organic base is sodium methoxide.

Embodiment 19. The method of embodiment 16, wherein the organic solvent is alcohol.

Embodiment 20. The method of embodiment 19, wherein the organic solvent is methanol.

Embodiment 21. A method of preparing a compound of Formula (I-g):

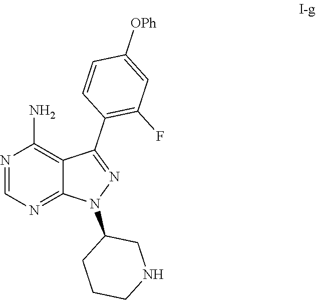 [SMILES: Nc1ncnc2c1c(-c1ccc(Oc3ccccc3)cc1F)nn2[C@@H]1CCCNC1]

or a salt thereof, by reacting a compound of Formula (I-f):

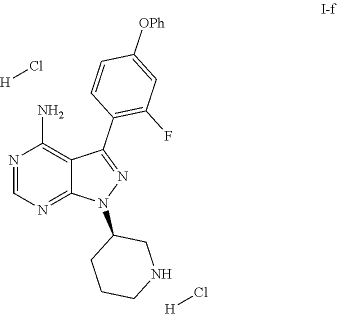

or a salt thereof, with base in alcohol and water to form the compound of Formula (I-g).

Embodiment 22. The method of embodiment 21, wherein the base is sodium hydroxide.

Embodiment 23. The method of embodiment 21, wherein the alcohol is methanol.

Embodiment 24. A compound of Formula (I-n):

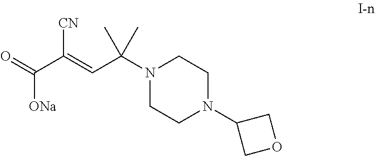

or a salt thereof.

Embodiment 25. An ester solvate of a compound of Formula (I-(E)):

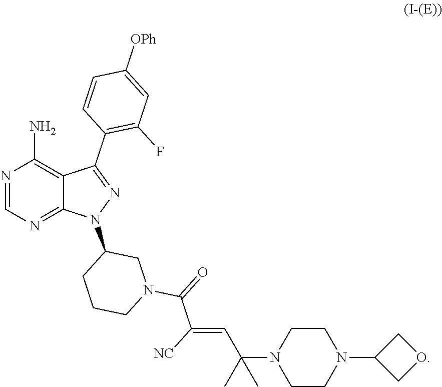 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

Embodiment 26. The solvate form of embodiment 25, wherein the ester solvate is a methylacetate solvate.

Embodiment 27. The solvate form of embodiment 26, wherein the methylacetate solvate has XRPD pattern comprising one or more peaks chosen from about 4.676° 2θ, 10.798° 2θ, 14.027° 2θ, 14.280° 2θ, 16.198° 2θ, and 16.704° 2θ.

Embodiment 28. A carbonate solvate of a compound of Formula (I-(E)):

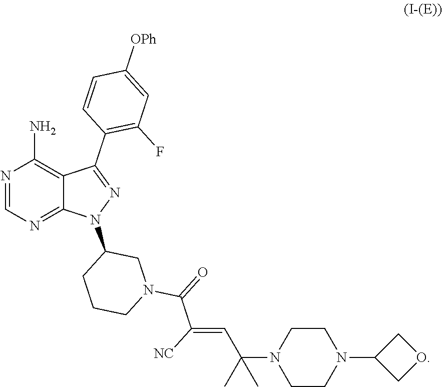 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

Embodiment 29. The solvate form of embodiment 28, wherein the carbonate solvate is a dimethylcarbonate solvate.

Embodiment 30. The solvate form of embodiment 29, wherein the dimethylcarbonate solvate characterized by an XRPD pattern comprising one or more peaks chosen from about 4.765° 2θ, 9.519° 2θ, 10.668° 2θ, 10.829° 2θ, 14.264° 2θ, and 16.976° 2θ.

Embodiment 31. A method of preparing the methylacetate solvate of embodiments 26 or 27, wherein crystalline Form B of the compound of Formula (I-(E)) is dissolved in methylacetate to form a solution.

Embodiment 32. The method of embodiment 31, wherein the solution is heated at a temperature ranging from 30 to 50° C. followed by cooling to a temperature ranging from 0 to 15° C.

Embodiment 33. A method of preparing the methylacetate solvate of embodiments 26 or 27, wherein the amorphous compound of Formula (I-(E)) is dissolved in methylacetate to form a solution.

Embodiment 34. The method of embodiment 33, wherein the solution is seeded with crystalline Form B of the compound of Formula (I-(E)) to form a suspension followed by stirring at a temperature ranging from 20 to 30° C.

Embodiment 35. A method of preparing the dimethylcarbonate solvate of embodiments 29 or 30, wherein crystalline Form C of the compound of Formula (I-(E)) is dissolved in dimethylcarbonate to form a solution.

Embodiment 36. The method of embodiment 35, wherein the solution is frozen, thawed, and stirred at a temperature ranging from 20 to 30° C. to induce particles that stay suspended in solution and do not sediment.

Embodiment 37. A method of preparing the dimethylcarbonate solvate of embodiments 29 or 30, wherein the amorphous compound of Formula (I-(E)) is dissolved in dimethylcarbonate to form a solution.

Embodiment 38. The method of embodiment 37, wherein the solution is seeded with crystalline Form B of the compound of Formula (I-(E)) to form a suspension.

Embodiment 39. The method of embodiment 38, wherein the suspension is frozen at a temperature ranging from −10 to −30° C.

Embodiment 40. The method of embodiment 39, wherein the frozen solution is thawed and stirred at a temperature ranging from 20 to 30° C.

Embodiment 41. A method of preparing a mixture of crystalline Form A and crystalline Form B of a compound of Formula (I-(E)) from the methylacetate solvate of embodiments 26 or 27, wherein the methylacetate solvate is crystallized in 2-10% dichloromethane in methylacetate with seeds of crystalline Form B, a mixture of crystalline Form A and crystalline Form B, or wet methylacetate solvate of the compound of Formula (I-(E)) to form a suspension.

Embodiment 42. The method of embodiment 41, wherein the methylacetate solvate is crystallized in 5-8% dichloromethane in methylacetate.

Embodiment 43. The method of embodiment 41, wherein the suspension is cooled, filtered, and dried to yield a mixture of crystalline Form A and crystalline Form B of the compound of Formula (I-(E)).

Embodiment 44. The method of embodiment 43, wherein the drying process is carried out at a pressure ranging from 0 to 800 mbars and a temperature ranging from 0 to 70° C.

Embodiment 45. A method of preparing crystalline Form B of the compound of Formula (I-(E)), wherein the suspension of embodiment 41 is partially dissolved in ethanol and subjected to heating and cooling cycles at a temperature ranging from 0 to 50° C.

Embodiment 46. The method of embodiment 45, wherein the temperature range of the heating and cooling cycles is from 20 to 43° C.

Embodiment 47. The method of embodiment 46, wherein the crystalline mixture is further cooled to a temperature of 0° C.

Embodiment 48. A method of preparing crystalline Form B of the compound of Formula (I-(E)), wherein the suspension of embodiment 41 is partially dissolved in isopropylacetate and subjected to heating, cooling, and stirring.

Embodiment 49. The method of embodiment 48, wherein the heating is carried out in 3-5 steps at a temperature ranging from 20 to 70° C. for 1 to 3 hours.

Embodiment 50. The method of embodiment 49, wherein the heating is carried out in 4 steps at a temperature ranging from 30 to 60° C. for 2 hours.

Embodiment 51. The method of embodiment 48, wherein the cooling is carried out at a temperature ranging from 15 to 40° C. within 1 to 3 hours followed by stirring for 8-16 hours.

Embodiment 52. The method of embodiment 51, wherein the cooling is carried out at a temperature ranging from 20 to 30° C. within 2 hours followed by stirring for 12 hours.

Embodiment 53. A method of preparing crystalline Form B of the compound of Formula (I-(E)), wherein the suspension of embodiment 41 is subjected to pressure.

Embodiment 54. The method of embodiment 53, wherein the pressure ranges from 30 to 70 bars.

Embodiment 55. The method of embodiment 53, wherein the pressure is applied with inert gas.

1. A method of preparing a compound of Formula (I): 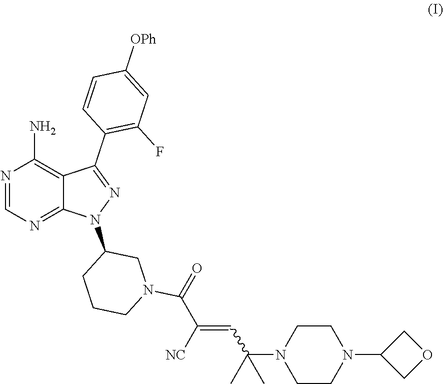 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]
or a salt thereof, comprising:
reacting a compound of Formula (I-g): 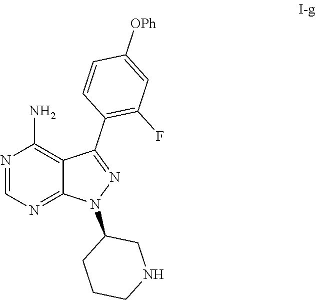 [SMILES: Nc1ncnc2c1c(-c1ccc(Oc3ccccc3)cc1F)nn2[C@@H]1CCCNC1]
or a salt thereof, with a compound of Formula (I-n): 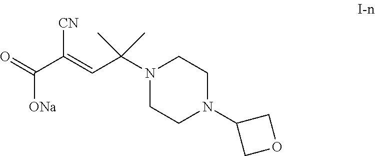
or a salt thereof, with amide-coupling reagent, organic base, and acid in organic solvent to form the compound of Formula (I).

2. The method of claim 1, wherein the amide-coupling reagent is propylphosphonic anhydride.

3. The method of claim 1, wherein the organic base is N-methylmorpholine, triethylamine, or diisopropylamine.

4. The method of claim 1, wherein the acid is hydrochloric acid, methanesulfonic acid, paratoluenesulfonic acid, or chlorotoluenesulfonic acid.

5. The method of claim 1, wherein the organic solvent comprises dichloromethane, dichloroethane, acetonitrile, chloroform, 2-methyltetrahydrofuran, tetrahydrofuran, methyl tert-butyl ether, toluene, chlorobenzene, methanol, ethanol, or isopropanol.

6. (canceled)

7. The method of claim 1, wherein the compound of Formula (I-n), or a salt thereof, is prepared by reacting a compound of Formula (I-l): 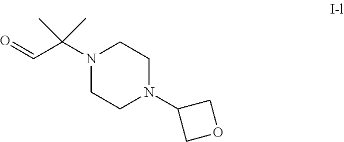
or a salt thereof, with a compound of Formula (I-m): 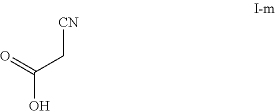
or a salt thereof, with inorganic or organic base in organic solvent to form the compound of Formula (I-n).

8. The method of claim 7, wherein the inorganic base is sodium hydroxide, lithium hydroxide, or potassium hydroxide.

9. The method of claim 7, wherein the organic base is sodium methoxide.

10. The method of claim 7, wherein the organic solvent is alcohol.

11. (canceled)

12. The method of claim 1, wherein the compound of Formula (I-g), or a salt thereof, is prepared by reacting a compound of Formula (I-f): 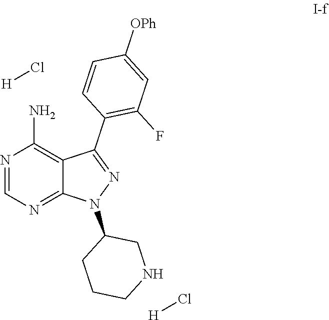
or a salt thereof, with base in alcohol and water to form the compound of Formula (I-g).

13. The method of claim 12, wherein the base is sodium hydroxide.

14. The method of claim 12, wherein the alcohol is methanol.

15. The method of claim 1, wherein the compound of Formula (I), or a salt thereof, is at least 95% by weight a compound of Formula (I-(E)): 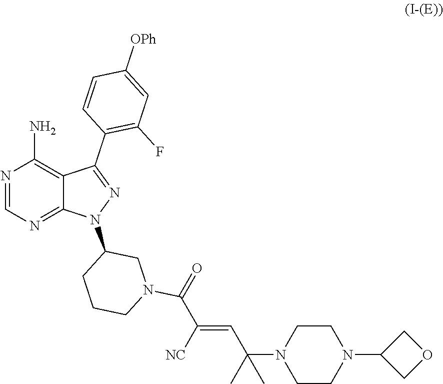 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]
or a salt thereof.

16. A method of preparing a compound of Formula (I-n): 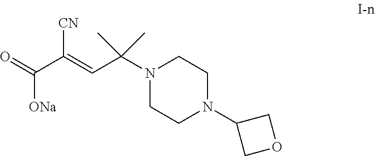
or a salt thereof, by reacting a compound of Formula (I-l): 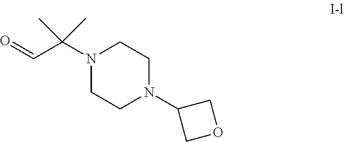
or a salt thereof, with a compound of Formula (I-m): 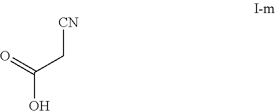
or a salt thereof, with inorganic or organic base in organic solvent to form the compound of Formula (I-n).

17. The method of claim 16, wherein the inorganic base is sodium hydroxide, lithium hydroxide, or potassium hydroxide.

18. The method of claim 16, wherein the organic base is sodium methoxide.

19. The method of claim 16, wherein the organic solvent is alcohol.

20. (canceled)

21. A method of preparing a compound of Formula (I-g): 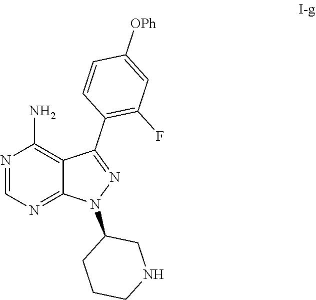 [SMILES: Nc1ncnc2c1c(-c1ccc(Oc3ccccc3)cc1F)nn2[C@@H]1CCCNC1]
or a salt thereof, by reacting a compound of Formula (I-f): 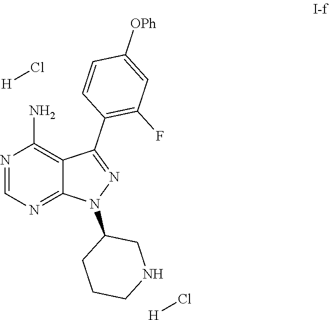
or a salt thereof, with base in alcohol and water to form the compound of Formula (I-g).

22. The method of claim 21, wherein the base is sodium hydroxide.

23. The method of claim 21, wherein the alcohol is methanol.

24. A compound of Formula (I-n): 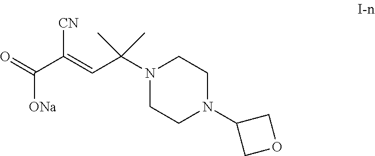
or a salt thereof.

25. An ester solvate of a compound of Formula (I-(E)):

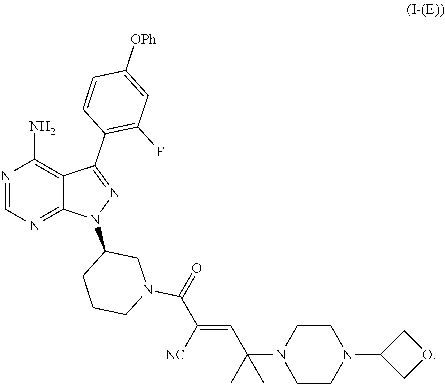 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

26. The solvate form of claim 25, wherein the ester solvate is a methylacetate solvate.

27. The solvate form of claim 26, wherein the methylacetate solvate has XRPD pattern comprising one or more peaks chosen from peaks at about 4.676° 2θ, 10.798° 2θ, 14.027° 2θ, 14.280° 2θ, 16.198° 2θ, and 16.704° 2θ.

28. A carbonate solvate of a compound of Formula (I-(E)):

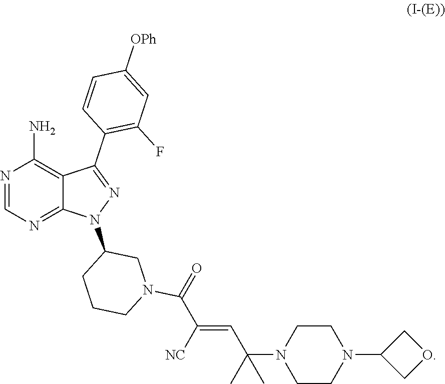 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

29. The solvate form of claim 28, wherein the carbonate solvate is a dimethylcarbonate solvate.

30. The solvate form of claim 29, wherein the dimethylcarbonate solvate characterized by an XRPD pattern comprising one or more peaks chosen from peaks at about 4.765° 2θ, 9.519° 2θ, 10.668° 2θ, 10.829° 2θ, 14.264° 2θ, and 16.976° 2θ.

31. A method of preparing the methylacetate solvate of claim 26, wherein crystalline Form B of the compound of Formula (I-(E)) is dissolved in methylacetate to form a solution.

32. The method of claim 31, wherein the solution is heated at a temperature ranging from 30 to 50° C. followed by cooling to a temperature ranging from 0 to 15° C.

33. A method of preparing the methylacetate solvate of claim 26, wherein the amorphous compound of Formula (I-(E)) is dissolved in methylacetate to form a solution.

34. The method of claim 33, wherein the solution is seeded with crystalline Form B of the compound of Formula (I-(E)) to form a suspension followed by stirring at a temperature ranging from 20 to 30° C.

35. A method of preparing the dimethylcarbonate solvate of claim 29, wherein crystalline Form C of the compound of Formula (I-(E)) is dissolved in dimethylcarbonate to form a solution.

36. The method of claim 35, wherein the solution is frozen, thawed, and stirred at a temperature ranging from 20 to 30° C. to induce particles that stay suspended in solution and do not sediment.

37. A method of preparing the dimethylcarbonate solvate of claim 29, wherein the amorphous compound of Formula (I-(E)) is dissolved in dimethylcarbonate to form a solution.

38. The method of claim 37, wherein the solution is seeded with crystalline Form B of the compound of Formula (I-(E)) to form a suspension.

39. The method of claim 38, wherein the suspension is frozen at a temperature ranging from −10 to −30° C.

40. The method of claim 39, wherein the frozen solution is thawed and stirred at a temperature ranging from 20 to 30° C.

41. A method of preparing a mixture of crystalline Form A and crystalline Form B of a compound of Formula (I-(E)) from the methylacetate solvate of claim 26, wherein the methylacetate solvate is crystallized in 2-10% dichloromethane in methylacetate with seeds of crystalline Form B, a mixture of crystalline Form A and crystalline Form B, or wet methylacetate solvate of the compound of Formula (I-(E)) to form a suspension.

42. The method of claim 41, wherein the methylacetate solvate is crystallized in 5-8% dichloromethane in methylacetate.

43. The method of claim 41, wherein the suspension is cooled, filtered, and dried to yield a mixture of crystalline Form A and crystalline Form B of the compound of Formula (I-(E)).

44. The method of claim 43, wherein the drying process is carried out at a pressure ranging from 0 to 800 mbars and a temperature ranging from 0 to 70° C.

45. A method of preparing crystalline Form B of the compound of Formula (I-(E)), wherein the suspension of claim 41 is partially dissolved in ethanol and subjected to heating and cooling cycles at a temperature ranging from 0 to 50° C.

46. (canceled)

47. The method of claim 45, wherein the crystalline mixture is further cooled to a temperature of 0° C.

48. A method of preparing crystalline Form B of the compound of Formula (I-(E)), wherein the suspension of claim 41 is partially dissolved in isopropylacetate and subjected to heating, cooling, and stirring.

49. The method of claim 48, wherein the heating is carried out in 3-5 steps at a temperature ranging from 20 to 70° C. for 1 to 3 hours.

50. (canceled)

51. The method of claim 48, wherein the cooling is carried out at a temperature ranging from 15 to 40° C. within 1 to 3 hours followed by stirring for 8-16 hours.

52. (canceled)

53. A method of preparing crystalline Form B of the compound of Formula (I-(E)), wherein the suspension of claim 41 is subjected to pressure.

54. The method of claim 53, wherein the pressure ranges from 30 to 70 bars.

55. The method of claim 53, wherein the pressure is applied with inert gas.
